(19) United States
(12) Patent Application Publication
(10) Pub. No.: US 2023/0125964 A1
(43) Pub. Date: Apr. 27, 2023

(54) Methods for high throughput cryopreservation of cell clusters

(71) Applicant: Regents of the University of Minnesota, Minneapolis, MN (US)

(72) Inventors: Li Zhan, St. Paul, MN (US); Joseph Sushil Rao, Minneapolis, MN (US); Zonghu Han, St. Paul, MN (US); Nikhil Sethia, Minneapolis, MN (US); Michael L. Etheridge, Maple Grove, MN (US); Cari S. Dutcher, North Oaks, MN (US); Erik B. Finger, Wayzata, MN (US); John C. Bischof, St. Paul, MN (US)

(21) Appl. No.: 17/969,979

(22) Filed: Oct. 20, 2022

Related U.S. Application Data
(60) Provisional application No. 63/270,192, filed on Oct. 21, 2021.

(51) Int. Cl. A01N 1/02; C12N 5/071

(52) U.S. Cl. CPC A01N 1/0221; C12N 5/0676; A01N 1/0284

(57) ABSTRACT

Methods for cryopreservation of biological samples are provided. The biological samples are sub-millimeter or millimeter scale biological materials. The biological samples are pancreatic islets and stem cell derived islets. Methods for cryopreservation of islets using cryomesh and multi-step loading and unloading of CPA cocktails are provided. Methods disclosed result in vitrified and rewarmed islets with high recovery, viability and functionality. Methods are scalable for high throughput production of large amounts of vitrified and rewarmed islets for use in therapeutic transplantation.

### CROSS-REFERENCE TO RELATED APPLICATION

The present application claims priority to and the benefit of U.S. provisional patent application Ser. No. 63/270,192 filed on Oct. 21, 2021, the entire contents of which are incorporated herein by reference.

This invention was supported by grants awarded by Regenerative Medicine Minnesota.

### BACKGROUND

Preservation of biological material is valuable in many areas including for medical and biological research. Diabetes has a tremendous adverse impact on the health, well-being, and longevity of affected individuals, as well as a considerable overall societal burden. Despite 100 years of therapeutic development since the discovery of insulin, current therapies, such as continuous glucose monitors, insulin pumps, and closed-loop systems, remain a treatment for the condition rather than a cure of the disease. While recent decades have seen substantial progress in the development of islet transplantation as a potential cure for diabetes, one of the main limitations of this approach is that transplants from a single donor are often insufficient to achieve insulin independence in the recipient. Frequently 2, 3, or more donor islet infusions totaling 700,000 to >1M islet equivalents (IEQ) are required for a “typical” 70 kg recipient, adding risks associated with repeat surgical interventions and multiple rounds of strong immunosuppression induction.

### SUMMARY

Pancreatic islet transplantation can cure diabetes but requires accessible, high-quality islets in sufficient quantities. Cryopreservation can solve islet supply chain challenges by enabling quality-controlled banking and pooling of donor islets. Approaches to islet cryopreservation must simultaneously provide high recovery, viability, function, and scalability. The present description includes methods for vitrification and rewarming (VR) of mouse, porcine, human, and human stem cell-derived (SC-beta) islets. The VR islets have been generated by comprehensive optimization of cryoprotectant agent (CPA) composition, e.g. CPA cocktail, CPA loading and unloading conditions. In one embodiment, the methods described herein can generate post-VR islet viability, relative to control, of about 90% for mouse, 92% for SC-beta, 87% for porcine, and 87% for human islets, and it remained unchanged for at least 9 months of cryogenic storage. VR islets can also have normal macroscopic, microscopic, and ultrastructural morphology. Mitochondrial membrane potential and ATP levels may be slightly reduced, but all other measures of cellular respiration, including oxygen consumption rate (OCR) to produce ATP, may be unchanged. VR islets may have normal glucose-stimulated insulin secretion function in vitro and in vivo. In the methods described herein, porcine and SC-beta islets can generate insulin in xenotransplant models, and mouse islets tested in a marginal mass syngeneic transplant model can cure diabetes in 92% of recipients within 24-48 hours following transplant. Excellent glycemic control can be seen for 150 days. In one embodiment, the methods described herein can be used to process 2,500 islets with >95% islets recovery at >89% post-thaw viability and can readily be scaled up for higher throughput. Cryopreservation may be used to supply needed islets for improved transplantation outcomes that cure diabetes.

In one aspect, the present description relates to a method for cryopreservation of a biological material. The method includes identifying a cryoprotective (CPA) cocktail for vitrification of a biological material. The CPA cocktail includes one or more CPAs at a vitrification CPA concentration for the biological material. The identity of the one or more CPAs and/or the vitrification CPA concentration are determined by analyzing the biophysical parameters of the biological material. The method includes loading the biological material with the one or more CPAs to attain the vitrification CPA concentration within the biological material. The method includes transferring the CPA loaded biological material onto a cryomesh or other porous surface and removing excess CPA cocktail surrounding the CPA loaded biological material prior to cooling the CPA loaded biological material. The method includes cooling the CPA loaded biological material on the cryomesh or the other porous surface to form a vitrified biological material and rewarming the vitrified biological material. The method includes unloading the one or more CPAs from within the rewarmed biological material to eliminate the one or more CPAs from a vitrified and rewarmed (VR) biological material.

The method may include minimizing the osmotic stress and/or chemical toxicity to the biological material during the loading, the cooling, the rewarming and/or the unloading steps. The biological material may include cell clusters. The biological material may include islets. The biological material may include human pancreatic islets, mouse pancreatic islets, porcine pancreatic islets, stem-cell derived islets, and/or stem-cell derived beta islets. The one or more CPAs in the CPA cocktail may include ethylene glycol (EG) and dimethyl sulfoxide (DMSO). The biophysical parameters of the biological material may include inactive volume fraction, hydraulic conductivity, and/or membrane permeability to the one or more CPAs. The chemical toxicity is minimized by decreasing the temperature of the biological material as the concentration of the one or more CPAs increases in the CPA cocktail. The osmotic stress on the biological material during loading the biological material with the one or more CPAs is minimized by gradually increasing the concentration of the one or more CPAs in the CPA cocktail to the vitrification CPA concentration prior to vitrification. The one or more CPAs in the vitrified biological material are unloaded by gradually decreasing the concentration of the one or more CPAs in the CPA cocktail after rewarming the vitrified biological material. The loading may include multi-steps and the concentration of the one more CPAs is increased in each successive loading step and the unloading may include multi-steps and the concentration of the one or more CPAs is decreased in each successive unloading step. The loading may include at least 3 steps and the unloading may include at least 3 steps. The loading may include increasing the concentration of the one or more CPAs in the CPA cocktail by a continuously flowing in the one or more CPAs gradually into the CPA cocktail. The unloading may include decreasing the concentration of the one or more CPAs in the CPA cocktail by diluting the CPAs in the CPA cocktail by continuously flowing in a diluent. The loading and unloading steps allow the biological material to gradually equilibrate to the surrounding CPA concentration to minimize osmotic stress. The vitrification CPA concentration in the CPA cocktail may be between about 42 weight percent of CPA and about 46 weight percent of CPA. The cooling rate for cooling the biological material may be greater than about 50,000° C./min. The rewarming rate for rewarming the vitrified biological composition may be greater than about 200,000° C./min. The unloading further includes gradually increasing the amount of a non-penetrating CPA in the CPA cocktail. The non-penetrating CPA may be sucrose. The sucrose may be increased from about 5% by weight to about 8.75% by weight. The duration of the loading steps and the unloading steps in a multi-step process may be between about 10 minutes and about 30 minutes and the duration of the unloading step may be between about 10 minutes and about 30 minutes. The total duration of the loading may be more than about 30 minutes and the total duration of the unloading may be more than about 30 minutes. The VR biological material may be allowed to recover prior to use for at least about 30 minutes after the unloading of the one or more CPAs. The VR biological material may have a viability of at least about 80% relative to a control. The VR biological material may have a cellular respiration, oxygen consumption rate to produce ATP, substantially similar to a control. The method may further include transplanting the VR biological material. The VR biological material may include greater than about 2500 islets with greater than about 95% recovery and greater than about 85% viability. The VR biological material may be VR Islets. The method may be scalable to produce at least 100,000 islets per batch.

In another aspect, the present description relates to a method of scaling up production of cryopreservation of biological material. The method includes identifying a cryoprotective (CPA) cocktail for vitrification of a biological material. The CPA cocktail includes one or more CPAs at a vitrification CPA concentration for the biological material. The identity of the one or more CPAs and/or the vitrification CPA concentration are determined by analyzing the biophysical parameters of the biological material. The method includes loading the biological material with the one or more CPAs to attain the vitrification CPA concentration within the biological material. The method includes transferring the CPA loaded biological material onto a cryomesh or other porous surface and removing excess CPA cocktail surrounding the CPA loaded biological material prior to cooling the CPA loaded biological material. The method includes cooling the CPA loaded biological material on the cryomesh or the other porous surface to form a vitrified biological material and rewarming the vitrified biological material. The method includes unloading the one or more CPAs from within the rewarmed biological material to eliminate the one or more CPAs from a vitrified and rewarmed (VR) biological material. The biological material may be cell clusters and the method produces at least about 10,000 cell clusters per batch. The two or more layers of the cryomesh or other porous surfaces may be stacked to increase the number of cell clusters per batch. The method may produce at least about 100,000 cell clusters per batch. The biological material may include islets.

In a further aspect, the present description relates to a method of therapeutic transplantation of a biological material. The method includes transplanting the biological material into a patient, wherein the biological material includes VR islets derived from one or more donors. The VR islets may have a viability of at least 80 percent. The biological material may include at least about 100,000 VR islets from one donor. The biological material may be from two or more donors and the biological material may include at least about 100,000 VR islets from each of the donors. The biological material may include at least about 300,000 VR islets, wherein the islets are pooled from two or more donors for transplantation to the patient.

In yet a further aspect, the present description relates to a vitrified and rewarmed biological composition. The composition includes cell clusters wherein the cell clusters have a recovery of at least about 90% and viability of at least about 80% relative to control. The VR biological material may have a cellular respiration, oxygen consumption rate to produce ATP that is substantially similar to a control. The cell clusters may have been cooled at a rate greater than about 50,000° C./min and rewarmed at a rate greater than about 200,000° C./min. The composition may include greater than about 2500 VR cell clusters. The composition may include greater than about 100,000 VR cell clusters from one donor. The composition may include greater than about 100,000 VR cell clusters pooled from two or more donors. The VR cell clusters may be VR islets.

In the following detailed description of illustrative examples, reference is made to specific embodiments by way of drawings and illustrations. These examples are described in sufficient detail to enable those skilled in the art to practice what is described, and serve to illustrate how elements of these examples may be applied to various purposes or embodiments. Other embodiments exist, and logical, mechanical, electrical, and other changes may be made.

Features or limitations of various embodiments described herein, however important to the examples in which they are incorporated, do not limit other embodiments, and any reference to the elements, operation, and application of the examples serve only to define these illustrative examples. Features or elements shown in various examples described herein can be combined in ways other than shown in the examples, and any such combinations is explicitly contemplated to be within the scope of the examples presented here. The following detailed description does not, therefore, limit the scope of what is claimed.

All patents, publications or other documents mentioned herein are incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B are schematic diagrams of the overview of the study and the cooling/warming rates of cryomesh.

FIG. 1C is a graph of a measured temperature profile of cryomesh VR islets.

FIG. 1D is a graph of a correlation of critical warming rate (CWR) and CPA concentration.

FIG. 1E is a schematic diagram of a cryopreservation method using cryomesh.

FIG. 2A is a schematic drawing of the microfluidic device used to measure the biophysical properties of the islets (not to scale). The morphological changes of the islets were recorded via a microscope.

FIG. 2B are images of shrinking/swelling of islets. The scale bar is 100 μm.

FIG. 2C are Boyle-van′ t Hoff plots of mouse and SC-beta islets.

FIG. 2D is a plot of normalized volume of mouse and SC-beta islets vs. time demonstrating the shrink-swell behavior when exposed to 15 wt % DMSO at 21° C. (n=5).

FIG. 2E is a summary table of mouse and SC-beta islets water (Lₚ) and CPA (ω) permeability at 4° C. and 21° C.

FIG. 2F is a plot of stepwise loading (step 1-3) and unloading (step 4-7) of 22 wt % EG+22 wt % DMSO for islets.

FIG. 2G is a plot of modeled islet normalized volume change during CPA loading/unloading using the measured biophysical properties.

FIG. 2H is a plot of modeled CPA concentration in the mouse and SC-beta islets.

FIG. 3A shows confocal microscope images of live and dead controls of SC-beta islets stained by Acridine Orange (AO, cyan color) and Propidium Iodide (PI, red color). The scale bar is 100 μm.

FIG. 3B shows confocal microscope images (AO/PI merge) of CPA treated (i.e., CPA loading and unloading only) and VR treated (i.e., CPA loading, VR, and CPA unloading) SC-beta islets. Various CPA formulations were examined. The scale bar is 100 μm.

FIG. 3C is a plot of cell viability of CPA only (cyan), VR (green) treated, and live control (red) SC-beta islets. Data are individual data points and mean±standard deviation (s.d.).

FIGS. 3D-3E are plots for mouse (FIG. 3D) and SC-beta (FIG. 3E) islet cell viability after different culture time (0, 3, 24 hrs) post-CPA exposure only and then post-VR treatment. Data are individual data points and mean±s.d.

FIG. 4A shows images of morphology of mouse islets from live control, VR (i.e., cryopreserved by VR), and conventional cryopreservation (i.e., cryopreserved by conventional slow freezing) evaluated by brightfield microscopy, H&E histology, and TEM. The scale bars are 2 μm for TEM, 50 μm for brightfield images, and 70 μm for histology.

FIG. 4B is a plot of viability (% of live control) of mouse, SC-beta, porcine, and human islets from treatment groups including live control, VR, VR 9 months (islets stored in LN₂ for 9 months prior to rewarming), conventional cryopreservation (cryopreserved by conventional slow freezing), and dead control (treated by 75% ethanol). Data are individual data points and mean±s.d.

FIG. 4C shows confocal microscope images (AO/PI) of mouse, SC-beta, porcine, and human islets from treatment groups, including live control, VR, and conventional cryopreservation. The scale bars are 100 μm.

FIG. 4D shows TUNEL-stained images of mouse, SC-beta, and human islets from treatment groups including live control, VR, and conventional cryopreservation. Bottom panel is Annexin V staining of mouse islets from the same treatment groups. The scale bars are 70 μm for TUNEL images and 100 μm for all fluorescence images.

FIG. 5A are images of mitochondrial membrane potential (via TMRE staining) of mouse, SC-beta, porcine, and human islets from treatment groups including live control, VR, and conventional cryopreservation. The scale bar is 100 μm.

FIG. 5B are plots with the left panel showing the quantification of TMRE staining intensity. Comparisons shown between live control and treatment groups were performed by Kruskal-Wallis and pairwise Wilcoxon tests (n=3-16/group). Right panel is the measurement of ATP levels of 4 types of islets from live and dead control groups and cryopreservation groups (i.e., VR and conventional cryopreservation). Data are individual data points and mean±s.d.

FIG. 5C is a plot of example oxygen consumption rate (OCR) curve showing the change in OCR during Mito Stress testing in SC-beta islets and comparing live control, VR, conventional cryopreservation, and dead control islets (n=3-4/group at each timepoint). Data are mean±s.d.

FIG. 5D are plots of compilation of the metabolic OCR parameters for each islet type and each treatment group. Data are individual data points and mean±s.d.

FIG. 5E are plots of in vitro glucose-stimulated insulin secretion (GSIS) assay for mouse, SC-beta, and human islets from treatment groups including live control, VR, and conventional cryopreservation. Data are individual data points and mean±s.d.

FIG. 6A is a plot of blood glucose levels of streptozotocin-induced diabetic mice after syngeneic transplant of marginal mass mouse islets (250 islets per recipient) from treatment groups including live control, VR, VR with 9-month cryopreserved storage (islets stored in LN₂ for 9 months), and conventional cryopreservation (450 islets per recipient). Data are mean±s.d.

FIG. 6B shows images of Insulin (red) and glucagon (green) staining in syngeneic (mouse) and xenogeneic (porcine and SC-beta) mouse transplant models. Treatment groups of islets include live control, VR, and conventional. DAPI is stained blue in the merged images. Scale bar is 200 μm.

FIG. 6C shows plots of intraperitoneal glucose tolerance testing (IPGTT) of wildtype mice, diabetic mice, and diabetic mice transplanted with live control, VR, and conventional cryopreserved islets (left panel). Area under the curve (AUC) of IPGTT (right panel). Data are individual data points and mean±s.d.

FIG. 6D is a plot of xenotransplant of SC-beta islets in non-diabetic NSG mice with non-fasting plasma human insulin levels at 4, 8, and 12 weeks posttransplant. Data are individual data points and mean±s.d.

FIG. 6E is a plot, at 14 weeks, of plasma insulin level of NSG mice after fasting and 30 minutes following stimulated insulin production by intraperitoneal glucose injection. Data are individual data points and mean±s.d.

FIG. 7 are plots showing in vitro differentiation of human stem cell (SC)-derived islets. SC-derived beta cell clusters (SC-beta) were differentiated from a human embryonic cell line (HUES8) in vitro.

FIG. 8 is a schematic diagram of 4 different Vitrification-Rewarming (VR) approaches tested for islet cryopreservation.

FIG. 9A shows plots at 21° C., the volume response of mouse islet (upper row) and SC-beta islet (lower row) to DMSO (left column), EG (middle column), and PG (right column).

FIG. 9B shows plots at 4° C., the volume response of mouse islet (upper row) and SC-beta islet (lower row) to DMSO (left column), EG (middle column), and PG (right column) The legends represent the diameter of islets.

FIG. 10A shows plots for short loading, step durations of 3 min, 11 min, and 11 min were used for the 1.3 M, 3.2 M, and 6.5 M steps, respectively. The final CPA chemical toxicity value (Jₜₒₓ, far right column) was computed to be 62.5.

FIG. 10B shows plots for medium loading, step durations of 10 min loading time were used for each 1.3 M, 3.2 M, and 6.5 M steps. The final CPA chemical toxicity value (Jₜₒₓ, far right column) was computed to be 60.2.

FIG. 10C shows plots for long loading, the step durations were 15 min, 35 min, and 5 min for 1.3 M, 3.2 M, and 6.5 M steps, respectively. The final CPA chemical toxicity value (Jₜₒₓ, far right column) was computed to be 73.2.

FIG. 11A shows plots of the number of TUNEL positive cells per islet which was measured for mouse, human, and SC-beta islets following VR vs. conventional cryopreservation and fresh control islets (n=9/group). Data are individual data points and mean±s.d.

FIG. 11B shows a plot of Annexin V translocation to the cell surface which was quantified by fluorescence intensity measurement for mouse islets of each treatment group (n=4/group). Kruskal-Wallis and pairwise Wilcox tests were used to compare groups. Data are individual data points and mean±s.d.

FIG. 12A shows confocal images of TMRE stained mouse islets. From left to right: live control, 0 hr post-VR, 3 hrs post-VR, and 24 hrs post-VR. Scale bars are 100 μm.

FIG. 12B shows plots of quantitative comparison of TMRE staining intensity from live control, 0 hr post-VR, 3 hrs post-VR, and 24 hrs post-VR islets. Error bars are mean±s.d.

FIG. 12C shows plots of initial oxygen consumption rate (OCR) of live control, 0 hr post VR, 3 hr post-VR, and 24 hr post-VR islets. Error bars are mean±s.d.

FIG. 12D shows plots of ATP content of live control, 0 hr post-VR, 3 hr post-VR, and 24 hr post VR islets. Error bars are mean±s.d.

FIG. 13 is a plot of blood glucose level before and after nephrectomy. Error bars are mean±s.d.

FIGS. 14A-14D is a schematic diagram of a prototype cryomesh scale up. FIG. 14A shows CPA is loaded in ramp or stepwise fashion while agitating the islet suspension. FIG. 14B shows islets are pumped onto individual discs (up to 60K IEQ/disc), and excess CPA is wicked. FIG. 14C shows discs are plunged into LN2 for vitrification and stored in a cryogenic storage system. FIG. 14D shows discs are plunged into rewarming solution, and CPA is offloaded as cells are prepared for transplant.

FIG. 15 is a graph of the recovery of islets post thaw using the VR approach described herein. Islet recovery is the ratio of islet quantity after cryopreservation to islet quantity before cryopreservation. For standard scale, 400-2,000 islets were used per test; for medium scale, 2,500 islets were used per test; for high scale, 10,000 islets were used per test. Data presented as mean±s.d. with individual measured datapoints. Number of independent experiments ranged from n=1 to n=26.

FIG. 16A and FIG. 16B are plots of beta cell specific viability in SC-beta islets. SC-beta islets were vitrified and rewarmed (VR), then dissociated to single cell suspensions, stained with fixable live/dead dye, fixed, permeabilized, and stained with anti-insulin antibodies. FIG. 16A are plots of representative gating scheme for flow cytometric analysis. An initial live/dead v. forward scatter demonstrated clear populations of live and dead cells. Following FSC/SSC and singlet gating a final 4-quadrant FACS plot showed individual populations of insulin+/− and live/dead cells. FIG. 16B is a plot from the FACS plots in FIG. 16A. The percent of live cells in the insulin positive and negative cell populations was determined (normalized relative to the input live control cells). One-way ANOVA with Tukey post hoc test was used to compare groups (n=3/group), and the p values are indicated. Data are individual data points and mean±s.d.

### DEFINITIONS

Various terms are defined herein. The definitions provided below are inclusive and not limiting, and the terms as used herein have a scope including at least the definitions provided below.

The terms “preferred” and “preferably”, “example” and “exemplary” refer to embodiments that may afford certain benefits, under certain circumstances. However, other embodiments may also be preferred or exemplary, under the same or other circumstances. Furthermore, the recitation of one or more preferred or exemplary embodiments does not imply that other embodiments are not useful, and is not intended to exclude other embodiments from the inventive scope of the present disclosure.

The singular forms of the terms “a”, “an”, and “the” as used herein include plural references unless the context clearly dictates otherwise. For example, the term “a tip” includes a plurality of tips.

Reference to “a” chemical compound refers one or more molecules of the chemical compound, rather than being limited to a single molecule of the chemical compound. Furthermore, the one or more molecules may or may not be identical, so long as they fall under the category of the chemical compound.

The terms “at least one” and “one or more of” an element are used interchangeably, and have the same meaning that includes a single element and a plurality of the elements, and may also be represented by the suffix “(s)” at the end of the element.

The terms “about” and “substantially” are used herein with respect to measurable values and ranges due to expected variations known to those skilled in the art (e.g., limitations and variability in measurements).

The terms “and/or” means one or all of the listed elements or a combination of any two or more of the listed elements.

The terms “comprises,” “comprising,” and variations thereof are to be construed as open ended—i.e., additional elements or steps are optional and may or may not be present.

Unless otherwise specified, temperatures referred to herein are based on atmospheric pressure (i.e. one atmosphere).

“Cryopreservation” as referred to herein relates to preservation of a biological sample/specimen at cryogenic temperatures. Cryopreservation includes cooling/freezing the biological sample below the freezing point of the sample in such a manner as to prevent significant damage due to ice crystallization and to a sufficiently low temperature in order to slow/arrest metabolic/chemical activity which can provide long term storage of biomaterials. Cryopreservation of a biological sample can also include warming the biological sample to recover the function/activity of the biological sample after storage at cryogenic temperatures.

“Cryogenic” or “cryogenic temperature” as referred to herein relates to a temperature below sub-zero. Cryogenic temperatures can be in the range from −80° C. (112° F.) to absolute zero (−273° C. or −460° F.), but includes any effects below the freezing point of the sample/specimen.

“Cryopreserved state” as referred to herein relates to a cryopreserved composition at a cryogenic temperature.

“Cryogenic coolant” as referred to herein relates to a substance that is at a cryogenic temperature, e.g. liquid nitrogen, slush nitrogen.

“Cryoprotective solution” or “CPA cocktail” as used herein relates to a solution that includes one or more cryoprotective agents (CPA). Cryoprotective solution may be referred to as a “CPA solution” or a “CPA cocktail”. “Cryoprotective solution”, “CPA solution” and “CPA cocktail” are used interchangeably herein.

“Vitrification CPA concentration” as used herein relates to the concentration of the CPA(s) that are present in the CPA cocktail when the biological material is cooled to a cryogenic temperature for vitrification. The vitrification CPA concentration can be determined to minimize injury to the biological material during vitrification and rewarming. The vitrification CPA concentration is determined based on the CPA thermophysical behavior, expected cooling and rewarming conditions, and the biophysical parameters of the biological material.

“Initial CPA concentration” as used herein relates to concentration of the CPA(s) that are present in the CPA cocktail when the biological material is first placed in a CPA cocktail for preparation for cryopreservation/vitrification. The initial CPA concentration may be zero.

“Osmotic stress” as used herein relates to shrinking and/or swelling of a biological sample when exposed to a solution, e.g. CPA cocktail. Osmotic stress can vary depending on the solution contents and can be minimized by gradually increasing or decreasing the contents of the solution gradually to allow the biological material to equilibrate to minimize the amount of shrinking and/or swelling of the biological sample.

“Cryobuffer” as referred to herein relates to an isotonic buffer that is used as the carrier solution for CPA and unloading solution to cryopreserve the biological sample.

“Cryotool” as referred to herein relates to a cryoresistant tool that can handle a biological sample. The cryotool can, for example, remove a sample from a cryogenic environment.

“Cryomesh” as referred to herein relates to a cryoresistant tool that can handle a biological sample. The cryomesh can, for example, retain a biological sample on the filaments of the mesh while enabling the removal of any cryoprotective solution surrounding the biological sample.

“Vitrification” as referred to herein relates to a biological sample that has attained a glassy, amorphous structure without significant ice crystallization when cryopreserved. Vitrified biospecimen can be stored at cryogenic temperatures (e.g. less than or equal to <150° C.) in an ice-free glassy state for indefinite periods of time. Vitrified samples, for example, may have less than 0.1% V/V of ice crystallization in the sample.

“Crystallized” sample as referred to herein relates to a biological sample that has attained some crystalline structure and may not produce a viable biological sample upon warming to room or physiological temperature. Crystallized samples may also be referred to herein as unvitrified samples, non-vitrified samples, or devitrified samples. These terms are used interchangeably herein.

“Vitrified and rewarmed” or “VR” as referred to herein relates to a vitrified and rewarmed biological material/sample. The VR sample may be used in biological/therapeutic application. Vitrification and rewarming may be performed in a variety of ways.

“High-throughput” as referred to herein relates to methods to rapidly process a large number of samples in short amount of time.

“Biological specimens” or “biological samples” or “biological material” or “biomaterials” are used interchangeably and as referred to herein relate to cells, germplasm, cell aggregates, islets, oocytes and the like. The germplasm can be from a variety of species including, for example, coral germplasm, mammalian germplasm, invertebrate germplasm and the like. The cell aggregates or cell clusters can include spheroids, organoids, hepatocyte clusters, 3-D cell clusters, stem-cell derived islets and the like. The biological samples can be unicellular organisms such as bacteria, protozoa and the like. The cell aggregates or islets and oocytes can be, for example, vertebrates such as fish, amphibians, mammals, humans and others and beta cells from invertebrates. The biological samples can be related to commercially relevant or endangered species (i.e. agriculture, aquaculture and biodiversity).

“Pancreatic islets” or “islets” as referred to herein relates to clusters of hormone producing cells from the pancreas. Islets can include any of the types of cells that produce hormones including Alpha cells, Beta cells, Delta cells, Epsilon cells, pancreatic polypeptide (PP) cells and the like. Islets can be derived from any number of sources including, for example, mice, porcine, human and the like. Islets may also be derived from stem cells (SC) such as SC-Beta cells. Stem cell derived islets or endocrine cell clusters may be derived from embryonic stem cells or induced pluripotent stem cells.

“Organoid” as referred to herein relates to a 3D multicellular in vitro tissue construct that mimics its corresponding in vivo organ such that it can be used to study aspects of that organ in culture or for therapeutic purposes.

“YR islets” as referred to herein relates to islets that have gone through the process of having been vitrified and then rewarmed. VR islets, for example, can be used for transplantation. VR islets may be from a single donor source or may be islets combined from multiple donors prior to and/or after VR. VR islets may be stem-cell derived islets from a single donor or from multiple donors.

“YR biological material” as referred to herein relates to biological material that has gone through the process of having been vitrified and then rewarmed. VR biological material, for example, can be used for transplantation. VR biological material, for example, can VR islets.

“Critical cooling rate” or “CCR” as referred to herein relates to the rate of temperature change required to cool a liquid to a stable vitrified state without forming significant ice.

“Critical warming rate” or “CWR” as referred to herein relates to the speed of temperature rise needed to avoid significant ice crystal formation and growth during rewarming of a vitrified liquid.

The term “sub-millimeter” sample as referred to herein relates to a biological sample that is equal to or less than about a millimeter.

The term “millimeter” sample as referred to herein relates to a biological sample that is equal to or more than about a millimeter.

Biological samples can include other components to aid in the cryopreservation process, e.g. cryopreserving agent, buffer or other media that are present when the biological sample is prepared, transferred and/or cryopreserved. The size of the biological sample may be characterized by the longest or shortest dimension of the biological sample or specimen.

### DETAILED DESCRIPTION OF ILLUSTRATIVE EMBODIMENTS

The present description is directed to systems and methods for cryopreservation of biological materials and rewarming of the cryopreserved biological materials. The present description includes methods for cryopreservation of sub-millimeter and/or millimeter scale biological materials. The present description can include a cryopreservation system that includes the use of a cryomesh in the cryopreservation protocols. The cryomesh can enable the retention of the biological material on the surface of the mesh and removal of cryoprotective agent surrounding the biological material prior to cryopreservation. Methods described herein include methods for cryopreserving the biomaterials with minimal to no cryoprotective agent solution surrounding the sample during vitrification. Methods include rewarming the cryopreserved sample that is viable for the desired end use. In one embodiment, the biomaterials that are cryopreserved using the methods described herein are pancreatic islets. In one embodiment, pancreatic islets are vitrified and rewarmed using the cryomesh in the methods described herein. The rewarmed islets may be used for therapeutic transplantation.

It will be understood that the present description will be described with respect to islets but the methods may be applied to other biological materials or samples and all are within the scope of this description.

Transplantation of a sufficient number of high-quality, viable, and functional pancreatic islets into a diabetic patient can cure this increasingly common and progressively debilitating disease. A significant limitation affecting the success of islet transplantation is the lack of an on-demand supply of sufficient numbers of high-quality native or stem cell-derived islets—a limitation that is exacerbated by the inability to store these cellular products prior to use.

Cryopreservation, or the stabilization of biomaterials at an ultralow temperature (<−150° C.), can achieve pooling, long-term banking, and off-the-shelf availability of viable cells and tissues. Conventional cryopreservation techniques utilize slow cooling (i.e., <1° C./min) of biological substances to a dehydrated frozen state with the presence of extracellular ice. The addition of low concentration (˜2 M) cryoprotectant agents (CPAs) helps stabilize cells during cryopreservation and improves cellular viability. Despite extensive investigation (Table 1), challenges persist for islet cryopreservation due to ice-related injury (i.e., suboptimal viability), inability to achieve clinical scalability, and use of non-clinically accepted components (i.e., fetal bovine serum) required to improve viability.

TABLE 1
Summary of islets cryopreservation studies

| | Cryopreservation details | | | | | Post cryopreservation results | | | |
|---|---|---|---|---|---|---|---|---|---|
| | | | | Cooling | War- | | | | |
| | Islet | | | rate | ming | Islet | | | |
| Ap- | quan- | Spe- | CFA | (° C./ | rate | recov- | Via- | | |
| proach | tity | cies | used | min) | (° C./min) | ery | bility | GSIS | Transport |
| Con- | NR | [symbol] | 0.5M EG + | 0.25 | −200 | 80-84% | 75-83% | 82-84% | Rat islets |
| ventional | | | 1M DMSO | | | | | | to inductive |
| (slow | | | | | | | | | monoglycerol |
| freezing) | | | | | | | | | in 4 days |
| | 500 | [symbol] | | | | | | | |
| | | encap- | 2M | 0.25 | −200 | 72-84% | 75-88% | 82-94% | monoglycerin |
| | | sulated | DMSO | | | | | | in 4 days |
| | | | | | | | | | node ride |
| | 50 | Rot | 15% | 0.5 | −200 | NR | 50% | 28% | NR |
| | | | DMSO | | | | | | |
| | 50,000 | [symbol] | 2M | 0.25 | −200 | 69% | 50% | NR | NR |
| | | | DMSO | | | | | | |
| | NR | Rat | 16% | 0.25 | −200 | 66% | 66% | −60% | NR |
| | | | DMSO | | | | | | |
| | 100 | Rat | 1.5M EG, | 1 | 35 | 85% | NR | 94.8% | [symbol] |
| | | | 5% hydroxy | | | | | | in 7 days |
| | | | [symbol] | | | | | | |
| | 500 | Mouse | 10% DMSO | 0.25 | −200 | 80% | NR | NR | [symbol] |
| | 10,000- | Rat | 2M | 0.25 | −200 | 78-79% | NR | 33-46% | Canine islets |
| | 33,000 | cog | DMSO | | | | | | to node mice: |
| | | | | | | | | | 62% |
| | | | | | | | | | achieved |
| | | | | | | | | | monoglycemia |
| | NR | Rat | 2M | 0.25 | 200 | NR | NR | NR | Monoglycemia |
| | | cog | DMSO | | | | | | delayed by |
| | | | | | | | | | several weeks |
| | 20 | Rat | 1M | variable | 7.5 | NR | NR | 57-100% | NR |
| | | cog | DMSO | | | | | | |
| | NR | Rat | 2.5-10% | 0.5-0.57 | 150 | NR | NR | NR | [symbol] |
| | | | DMSO | | | | | | |
| Verifi- | 190 | Rat | [symbol] | NR | NR | 94-97% | 70% | 65% | [symbol] |
| cation | | | DMSO | | | | | | |
| and | 10-50 | Rat | [symbol] | NR | NR | 99-100% | 64% | 38% | NR |
| re- | | | [symbol] | | | | | | |
| warming | 25-35 | Mouse | [symbol] | NR | NR | NR | NR | 13% | Monoglycemia |
| (VR) | | | DMSO | | | | | | in mice |
| | | | | | | | | | in 4-8 days |
| | 10 | Rart | 15% EG-15% | 23,000 | 42,000 | NR | 37% | 58% | NR |
| | | | DMSO 0.5M | | | | | | |
| | | | sucrose | | | | | | |
| | 100 | Rat | 30% EG- | NR | NR | NR | 85% | 91% | [symbol] [symbol] |
| | | | 20% DMSO | | | | | | in 2 days |
| | | | [symbol] | | | | | | |
| | 500 | Rat | DP6 | 43 | 225 | 70% | NR | NR | NR |
| | 50 | [symbol] | [symbol] | 43 | 500 | NR | NR | NR | [symbol] |
| | 10,000 | [symbol] | [symbol] | NR | −200 | 95% | 17% | NR | NR |
| | 50,300 | Mouse | [symbol] | NR | −200 | 51-61% | 65% | −65% | [symbol] |
| | 50 | [symbol] | [symbol] | NR | −200 | 80% | 85% | 44% | NR |
| | [symbol] | [symbol] | [symbol] | [symbol] | [symbol] | [symbol] | [symbol] | [symbol] | [symbol] |

Summary of islets cryopreservation studies

| | | | | Scalability | |
|---|---|---|---|---|---|
| Ap- | Recovery | Viability | Function* | >1,000 | |
| proach | ≥90% | ≥85% | ≥90% | islets | Reference |
| Con- | ϰ | ϰ | ✓ | ϰ | Kojoyata, |
| ventional | | | | | 2019 |
| (slow | ϰ | ϰ | ✓ | ϰ | Kojoyao, |
| freezing) | | | | | 2019 |
| | ϰ | ϰ | ϰ | ϰ | Yotonaka, |
| | | | | | 2016 |
| | ϰ | ϰ | ϰ | ϰ | Mirotuda, |
| | | | | | 2013 |
| | ϰ | ϰ | ϰ | ϰ | Taylor, |
| | | | | | 2009 |
| | ϰ | ϰ | ✓ | ϰ | Miyamoto, |
| | | | | | 2001 |
| | ϰ | ϰ | O | ϰ | Cotbal, |
| | | | | | 1998 |
| | ϰ | ϰ | O | ✓ | LAkey, |
| | | | | | 1996 |
| | ϰ | ϰ | O | ϰ | Rajone, |
| | | | | | 1984 |
| | ϰ | ϰ | O | ϰ | Bank, |
| | | | | | 1981 |
| | ϰ | ϰ | O | ϰ | Rajote, |
| | | | | | 1977 |
| Verifi- | ✓ | ϰ | O | ϰ | Nakayoma, |
| cation | | | | | 2020 |
| and | ✓ | ϰ | ϰ | ϰ | Yonaonaka, |
| re- | | | | | 2017 |
| warming | ϰ | ϰ | O | ϰ | Nagoya, |
| (VR) | | | | | 2016 |
| | ϰ | ϰ | ϰ | ϰ | Yamanaka, |
| | | | | | 2016 |
| | ϰ | ✓ | ✓ | ϰ | Sosomoda, |
| | | | | | 2012 |
| | ϰ | ϰ | ϰ | ϰ | Taylor, |
| | | | | | 2009 |
| | ϰ | ϰ | O | ϰ | Agulelo, |
| | | | | | 2008 |
| | ✓ | ϰ | ϰ | ✓ | Langet, |
| | | | | | 1999 |
| | ϰ | ϰ | O | ϰ | Jaue, |
| | | | | | 1987 |
| | ϰ | ✓ | ϰ | ϰ | Jaue, |
| | | | | | 1987 |
| | ✓ | ✓ | ✓ | ✓ | ✓ |

“✓” Indicates GSIS >90% and transplant achieves nomoglycemia;
“O” Indicates either GSIS >90% or transplant achieves nomoglycemia;
“ϰ” Indicates GSIS <90% (or NR) nad transport fails to achieve nomoglycemia for NR)
NR: not reported;
CPA: cryoprotectent agent;
DMSO: dimethyl sulfoxide;
EG: ethylene glycol;
PG: propylene glycol;
GSIS: glucose-stimulated Insulin Secretion. [symbol]
indicates data missing or illegible when filed

In some embodiments, the present description can include an effective method for long-term islet preservation that achieves high viability, recovery, function, and scalability. The method can include pancreatic islet banking by cryopreservation that could revolutionize the supply chain for islet isolation, allocation, and storage prior to transplant, increasing the overall utilization of deceased donor pancreases and curing more patients of diabetes.

High-throughput cryopreservation of biological material, for example, islets, can be performed using the systems and methods described herein. Well-established, reproducible cryopreservation of biological material can provide a unique opportunity to preserve and expand the use of important biological material.

In some embodiments, the cryopreservation methods described herein can include ice-free vitrification—rapid cooling of a biomaterial to a glass-like state. To avoid ice formation, the cooling and subsequent warming rates can exceed the critical cooling rate (CCR) and critical warming rate (CWR), respectively.

Increasing CPA concentration (>4 M) can lower the required CCR and CWR to attainable levels, but can cause toxicity in cells and tissues, especially at higher temperatures (>4° C.). Without being bound by any theory, a critical balance point must be found to avoid both injury from ice and from CPA toxicity, all while maintaining viability, functionality and clinical scalability. Previous islet vitrification strategies were limited to small volumes with low islet quantities (<150 islets in microliter volumes of CPA solution, Table 1) to ensure sufficient cooling and warming rates. To achieve clinical throughput of these prior approaches, volumetric scale-up reduces the cooling and warming rates and leads to ice formation, compromising viability.

Cryopreservation can allow viable cells and tissues to be preserved over time in the hypothermic, frozen, or vitrified (glassy) state. This disclosure describes systems, compositions and methods that may be used to cool biological samples and rewarm cryopreserved biological samples from cryogenic temperatures. The systems, methods and compositions described herein are useful in, for example, cooling sub-millimeter- or millimeter-scale cryopreserved biological samples such as, for example, islets and the like. The cryopreservation systems described herein advantageously can be used in methods to process biological samples for long-term storage by cryopreservation and also rewarming of the cryopreserved material. High-throughput techniques can be adapted for scalability in processing a large number of samples during cryopreservation and rewarming.

In the methods described herein the cryopreservation performance can be maximized by ice avoidance, by minimizing osmotic stress on the biological material and by minimizing CPA chemical toxicity during the VR processing of the islets for transplantation. The methods described herein can include optimizing the CPAs and the concentration of the CPAs during vitrification, e.g. the vitrification CPA concentration, by analyzing the biophysical parameters of the biological material. The biophysical parameters analyzed can include, for example, determining the inactive volume fraction of the biological material, hydraulic conductivity, membrane permeability to CPAs, and temperature- and rate-dependent toxicity of the CPAs.

The methods described herein can include optimizing vitrification CPA concentration, identifying the effective CPAs, loading methods, unloading methods, vitrification optimization by enhancing cooling and rewarming rates to develop a new islet cryopreservation method that can simultaneously achieve high recovery, viability, functionality, and scalability. In some embodiments, the methods can include vitrification and rewarming of mouse, porcine, human, and human SC-derived islets. Extensive in vitro assessments of viability, metabolic health, and insulin secretion were performed on the VR islets.

In some embodiments, the methods described herein can include syngeneic and xenogeneic transplantation of islets after vitrification and rewarming. The islets can remain viable in vivo, produce insulin, respond to glycemic challenge, and cure diabetes.

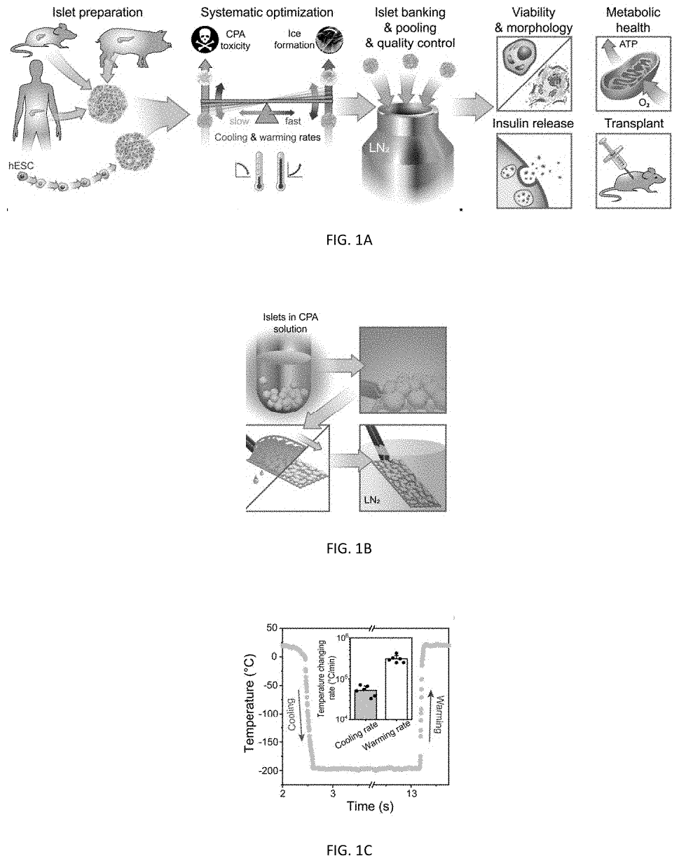

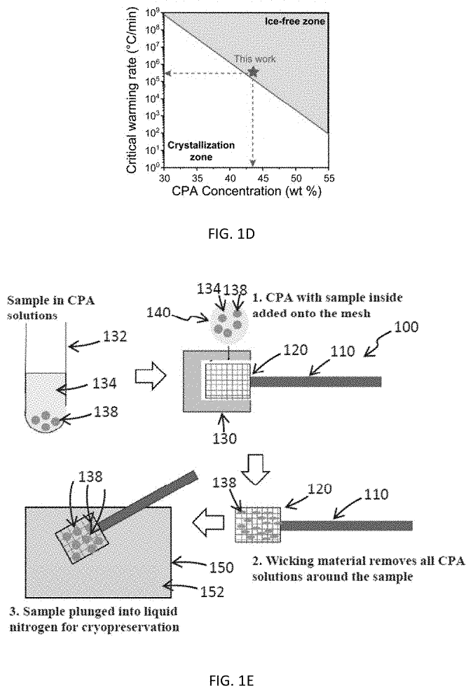

FIGS. 1A-1D illustrate one embodiment of an overview of the methods described herein for cooling/warming islets using cryomesh. Cryopreservation can be the cornerstone of an islet supply chain, allowing pooling, banking, and quality control prior to transplant. FIG. 1A shows model systems that can include, for example, mouse, porcine, human islets, and human embryonic stem-cell-derived beta cell clusters (referred to as SC-beta islets). To achieve high recovery, viability, function, and scalability simultaneously, systematic optimization of interrelated parameters including cryoprotectant agent (CPA) toxicity, ice formation, and cooling and warming rates during vitrification and rewarming (VR) cryopreservation can be performed in these islet systems. The achieved cooling and warming rates can be adjusted to balance the CPA toxicity and ice formation. Islet morphology, viability, metabolic health, in vitro, and in vivo function can be evaluated post VR cryopreservation. FIG. 1B shows a schematic diagram of cryomesh VR (not to scale). After CPA loading, islets in suspension can be transferred to the cryomesh, and excessive CPA solution can be removed before being plunged into liquid nitrogen (LN₂). FIG. 1C shows a measured temperature profile of cryomesh VR of islets. Inset is the achieved cooling and warming rates. FIG. 1D is a graph of a correlation of critical warming rate (CWR) and CPA concentration as described herein and in comparison to prior art demonstrating failure due to ice crystallization. FIG. 1D indicates that about 44 wt % CPA can be minimally required to avoid lethal ice using cryomesh VR and shows where other studies have failed to use a CPA with an adequate CWR to avoid ice under their thermal performance conditions.

The methods described herein can include a holistic, integrative optimization of physical and biological parameters to improve VR islets outcomes and address the supply chain challenges for high quality and quantity of pancreatic islets for therapeutic applications. The CPA concentration and formulation, loading and unloading procedure, and cooling and warming rates can be carefully balanced. In some embodiments the method may exceed the CCR of the CPA used. In some embodiments, the method can avoid damaging ice formation during rewarming and the method can exceed the CWR of the CPA at the concentration (wt %) used.

The systems and methods described herein can preserve and restore the integrity of the biological samples upon rewarming. The cooling of the biological sample can result in vitrification of the sample. In one embodiment, this description is directed to systems and methods that can include cooling that can achieve sufficiently high cooling rates that may exceed the critical cooling rates (CCRs) of the CPAs to produce VR islets suitable for applications such as therapeutic transplantation.

In some embodiments, the present description can include a cryopreservation system. The cryopreservation system can include a cryomesh tool for the cryopreservation of a biological sample as described below. In some embodiments, cryomesh can include a handle and a mesh attached to the handle. Advantageously, the cryomesh is a simple, versatile platform that can be used for high throughput cryopreservation (cooling and rewarming) of biological samples, e.g. biological samples in the sub-millimeter or millimeter range, and which can provide capability for rapidly increased cooling and rewarming rates over currently applied approaches.

FIG. 1E shows one embodiment of a cryopreservation system and method that includes cryomesh 100. Cryomesh 100 can include handle 110 and mesh 120. Handle 110 can be made from a variety of materials. The handle material can be cryoresistant. The material may be rigid. The material may be sufficiently rigid to hold the mesh in place when the biological sample is placed on the mesh. Handle 110 may be made from, for example, plastics, wood, metal and the like. Handle 110 can include plastics such as acrylics, polyesters, nylons, silicones, polyurethane, halogenated plastics, polyethylene, polypropylene, polystyrene, polyvinyl chloride and the like. In one embodiment, handle 110 is made of a plastic.

The length of handle 110 can vary and can be dependent on the specific need and the desired use. Any length of handle 110 may be used for cryomesh 100. In some embodiments, the length of handle 110 can be between about one inch and about 48 inches. In some embodiments, the length of handle 110 can be between about 6 inches and 36 inches; or between about 12 inches and about 24 inches. Handles outside of these ranges are also within the scope of this description.

Cryomesh 100 can be assembled by purchasing the handle, for example, from Thermo Fisher Scientific in Waltham, Mass. and purchasing the mesh, e.g. nylon mesh, for example, from Amazon.com in Seattle, Wash.

Mesh 120 can be permanently and/or removably attached to handle 110 of cryomesh 100. As shown in FIG. 1E, mesh 120 can be attached to handle 110 in a manner that mesh 120 can retain biological sample 138 on mesh 120 when cryoprotective agent (CPA) solution 134 surrounding sample 138 is removed. Mesh 120 is a porous mesh. The gaps within mesh 120 are sized such that the biological specimen placed on mesh 120 will not pass through the gaps but will be retained on mesh 120. CPA solution 134 can be substantially removed or wicked away from biological sample 138 by a variety of methods. In one embodiment, CPA solution 134 is removed from sample 138 by wicking solution 134 by wicking material 130. Wicking material 130 can be, for example, wicking paper. In one embodiment, CPA solution 134 may also be removed by the use of an external vacuum.

The characteristics of mesh 120 can vary and can be selected depending on the desired use of cryomesh 100 and biological sample 138. In one embodiment, mesh 120, for example, can vary depending on the size and nature of the biological sample. The characteristics of mesh 120 can impact the ability of biological sample 138 to adhere to and/or be retained on mesh 120. The characteristics of mesh 120 can affect the density of specimen that can be packed onto mesh 120. The characteristics of mesh 120 can affect the ability to wick away excess CPA solution 134.

The characteristics of mesh 120 can vary depending on the materials, mesh patterns, mesh density, mesh filament geometry, mesh filament surface and the like, and may also include a porous surface. Materials for mesh 120 can include, for example, plastics, metals, nylon, carbon elastomer and the like. Plastics can include, for example, acrylics, polyesters, silicones, polyurethane, halogenated plastics, polyethylene, polypropylene, polystyrene, polyvinyl chloride, graphite, polydimethylsiloxane and the like. Mesh may include other natural and manmade polymers and all are within the scope of this description. Mesh may also include metals such as, for example, aluminum, copper, stainless steel and the like.

Mesh 20 can include a variety of sizes for the openings between the filaments within mesh 20. The size of the openings can vary and can be dependent on the size of the biological sample that is cryopreserved. In one embodiment, the size of the openings is less than about one millimeter; or less than about 750 micrometers; or less than about 500 micrometers; or less than about 250 micrometers; or less than about 100 micrometers; or less than about 50 micrometers; or less than about 10 micrometers.

In some embodiments, the openings in the mesh can be greater than about one micrometer; or greater than about 50 micrometers; or greater than about 100 micrometers; or greater than about 250 micrometers; or greater than about 500 micrometers; or greater than about 750 micrometers; or greater than about 900 micrometer.

In one embodiment, the openings in the mesh can be between about 10 micrometers and about 100 micrometers; or between about 10 micrometers and about 50 micrometers.

Patterns for mesh 120 can include, for example, plain weave, twill weave, dutch weave and the like. The density of mesh 120 can include, for example, a range from about 50 to about 1250 mesh per inch. In some embodiments, the density of mesh 120 can be between about 100 mesh per inch and about 1000 mesh per inch; or between about 250 mesh per inch and about 500 mesh per inch. The filament geometry of mesh 120 can include, for example, cylindrical, rectangular and the like. In some embodiments, mesh filament surfaces can include, for example, hydrophilic surfaces. In some embodiments, mesh filament surfaces can include, for example, hydrophobic surfaces.

In some embodiments, the mesh size can impact the total amount of biological specimen that can be cryopreserved. In some embodiments, the length of the mesh can be between about 1 cm and about 30 cm; or between about 5 cm and about 20 cm; or between about 10 cm and about 15 cm. Other lengths outside of this range are also within the scope of this description.

In some embodiments, the width of the mesh can be between about 1 cm and about 30 cm; or between about 5 cm and about 20 cm; or between about 10 cm and about 15 cm. Other widths outside of this range are also within the scope of this description.

In some embodiments, the thickness of the mesh can be between about 0.01 mm and about 0.1 mm; or between about 0.1 and about 0.3 mm; or between about 0.3 and about 0.5 mm Other thicknesses outside of this range are also within the scope of this description.

The mesh can be in a variety of shapes and all are within the scope of this description. In some embodiments, the mesh is in the shape of a square, a rectangle, a circle and the like.

In some embodiments, cryomesh 100 can be incorporated into an automated or high throughput or “assembly-line” type approach (e.g. a continuous length or coiled cryomesh). In some embodiments, the scalability of the cryopreserved samples can be increased by increasing the width and/or the length of the cryomesh. In some embodiments, the scalability of the cryopreserved samples can be increased by stacking a number of cryomesh to accomplish a high-throughput approach as shown, for example, in FIGS. 14A-14D. Each layer of cryomesh is separated sufficiently within the cryomesh stack to achieve the desired CCRs and CWRs. In some embodiments, the cryomesh in a stack may be cooled and rewarmed individually, to achieve the desired CCRs and CWRs. Other methods of increasing scalability by increasing the amount of cryomesh available to hold the islets may be used and are within the scope of this description.

In some embodiments, the characteristics of mesh 120, e.g. mesh pattern, mesh density, filament geometry (e.g. shape, size), material and the like, can impact the cooling rates experienced by the loaded biological specimen under convective cooling. The cooling rate, for example, can be impacted through exposed surface area, biological specimen contact area, and heat transfer characteristics of mesh 120.

In some embodiments, the material and geometry of the mesh can be designed for low thermal mass (mass of the mesh*heat capacity of the mesh material) and high thermal conductivity. The contact area between the biomaterial and the mesh can be increased. Those combined conditions can lead to desired faster cooling/warming rate.

In some embodiments, the characteristics of mesh 120, e.g. mesh pattern, mesh density, filament geometry (e.g. shape, size), material and the like, can impact the cooling and/or rewarming experienced by the loaded biological specimen under convective rewarming. The rewarming rate, for example, can be impacted through exposed surface area, biological specimen contact area, and heat transfer characteristics of mesh 120.

Without being bound by any theory, the desired success across a range of biological specimen may require optimization of the cryomesh design parameters to achieve the required loading, cooling, and rewarming rates for specific applications.

A variety of biological samples can be cryopreserved according to the systems and methods described herein. In some embodiments, biological samples can be islets from terrestrial and/or aquatic organisms. In some embodiments, biological samples can be pancreatic islets. While described herein in the context of an exemplary embodiment in which the biological samples are islets or stem-cell derived islets, the systems and methods described herein can be applied to a variety of biological materials such as, for example, cell clusters, spheroids, organoids, hepatocyte clusters, 3-D cell clusters, stem-cell derived islets and the like.

In some embodiments, the present description can include a method for cryopreservation of biological materials. In one embodiment, the biological materials are islets. The method can include obtaining the biological material to be cryopreserved. In one embodiment, the biological material can be isolated and cultured from tissues and placed in a desired and/or a suitable media or buffer. The biologic material can be cells or cell clusters suspended in a solution. The biological material may be in, for example, a buffer for maintaining the biological material prior to cryopreservation. The biological material may be at a stage, e.g. a fully differentiated state, desired for cryopreservation. In one embodiment, the biological material may be stem cell derived material that is fully differentiated.

The biological material can be a variably sized biomaterial specimen. The biological material can be any sub-millimeter- or millimeter scale biomaterial. In some embodiments, the term sub-millimeter- or millimeter scale sample can have a largest linear dimension of less than about ten millimeters (mm); or less than about five mm; or less than about one mm; or less than about 0.9 mm; or less than about 0.7 mm; or less than about 0.5 mm; or less than about 0.3 mm; or less than about 0.1 mm; or less than about 50 micrometers; or less than about 10 micrometer; or less than about 1 micrometer.

In some embodiments, the term sub-millimeter- or millimeter scale sample can have a smallest linear dimension of greater than about one micrometer; or greater than about 10 micrometer; or greater than about 0.1 mm; or greater than about 0.3 mm; or greater than about 0.5 mm; or greater than about 0.7 mm; or greater than about 0.9 mm; or greater than about one mm; or greater than about five mm; or greater than about ten mm.

In one embodiment, the biological material can be between about 50 micrometers and about 500 micrometers. Biological materials outside of this range are also within the scope of this description.

The methods described herein can include loading the biological material, e.g. the islets, with a CPA solution. The CPA solution can include one or more cryoprotective agents.

While described herein in the context of an exemplary embodiment in which the cryoprotective agent includes ethylene glycol (EG) and dimethyl sulfoxide (DMSO), the composition, systems and methods described herein can involve the use of other one or more suitable cryoprotective agents and all are within the scope of this description. Exemplary suitable cryoprotective agents include, but are not limited to, combinations of alcohols, sugars, polymers, and ice blocking molecules that alter the phase diagram of water and allow a glass to be formed more easily (and/or at higher temperatures) while also reducing or controlling the likelihood of ice nucleation and growth during cooling or thawing. In some embodiments, cryopreservative agents may not be used alone, but in combination with other CPA and/or suitable agents that promote cryopreservation. In the case of vitrification solutions, exemplary cryopreservative cocktails are reviewed, for example, in Fahy et al., incorporated herein by reference. Additional exemplary cryopreservative solutions can include one or more of the following: dimethyl sulfoxide, glycerol, propylene glycol, ethylene glycol, sucrose, trehalose, raffinose, polyvinylpyrrolidone, and/or other polymers (e.g., ice blockers and/or anti-freeze proteins). Other cryopreservative agents may be included in the cryopreservative solutions and all are within the scope of this description.

The cryoprotective agents may be penetrating cryoprotective agents such as, for example, EG, DMSO, PG, methanol, glycerol, formamide, and the like. Non-penetrating cryoprotective agents may also be used during vitrification and/or rewarming. Non-penetrating cryoprotective agents can be, for example, sucrose, trehalose, lactose, sorbitol, Ficoll, polyethylene glycol (PEG), polyvinyl pyrrolidone (PVP), polyvinyl alcohol, polyglycerol, and the like. Penetrating CPAs are CPAs that are able to enter the intracellular space of the biological system of interest, while non-penetrating CPAs remain extracellular.

The cryoprotective agent(s) may be present in the CPA cocktail at various concentrations. The amount or concentration of CPAs in the CPA cocktail during vitrification can be referred to as the vitrification CPA concentration. In some embodiments, the vitrification CPA concentration and VR processing of the biological material can be determined by analyzing the biophysical parameters of the biological material to minimize the chemical toxicity of the CPAs and to minimize the osmotic stress on the biological material. VR processing can include, for example, loading the biological material with CPAs, cooling the loaded biological material, rewarming the vitrified biological material and unloading CPAs after the rewarming of the vitrified biological material. The biophysical parameters can include determining the inactive volume fraction of a biological material. The biophysical parameters can also include determining the hydraulic conductivity and the membrane permeability of CPAs. The biophysical parameters can include the temperature- and rate-dependent toxicity of the CPA. Descriptions on the study of each are described in the Methods below. In some embodiments, the water permeability can be, for example, between about 2×10⁻¹⁵ to 3×10⁻¹³ m/s*Pa. Water permeability outside of this range is also within the scope of this description. In some embodiments, the CPA permeability can be, for example, between about 7.5×10⁻¹² to 8×10⁻¹⁰ mol/Pa*m²*s. CPA permeability outside of this range is also within the scope of this description. In some embodiments, the osmotic inactive volume can be, for example, between about 0.2 and about 0.6. Osmotic inactive volume outside of this range are also within the scope of this description.

In some embodiments, the CPAs may be present, for example, at a molarity of no more the 8 M such as, for example, no more than 6 M, no more than 5 M, for example, no more than 4 M, for example, no more than 3 M, for example, no more than 2 M, for example, no more than 1 M, for example, no more than 900 mM, for example, no more than 800 mM, for example, no more than 700 mM, for example, no more than 600 mM, for example, no more than 500 mM, or for example, no more than 250 mM.

In some embodiments, the vitrification CPA concentration, may be, for example, at a weight percent of no more than about 50% by weight, or no more than 45% weight, or no more than 40% weight, or no more than 30% by weight, or no more than 20% by weight, or no more than 10% by weight.

The method can include loading the biological material with CPAs. Loading as referred to herein relates to exposing the biological material to CPAs to achieve the desired concentration of the CPA cocktail in the biological material prior to cooling the biological material to a cryogenic temperature. In some embodiments, the method can include loading the biological material with the CPAs by gradually increasing the concentration of the CPAs in the CPA cocktail. In some embodiments, the gradual increase may be achieved in a multi-step process. In some embodiments, the gradual increase may be achieved in a continuous flow addition of the CPAs as described below.

In some embodiments, the CPA concentration may be increased in a multi-step manner of increasing the concentration of the CPA concentration in each successive step until the vitrification CPA concentration is achieved. In some embodiments, the method can include loading the biological material, e.g. islets, with a CPA cocktail in a stepwise manner of a multi-step protocol. In other words, the biological material can be exposed to lower concentrations of the cryoprotective agent(s) in the first step and the concentration of the CPA in the CPA cocktail can be increased in each successive step until the desired cryoprotective concentration is loaded into the biological material in the final step. The temperature at which the loading of the CPA cocktail is performed can also be decreased in a stepwise manner as the concentration of the CPA increases in the CPA cocktail.

In some embodiments, the amount of CPA in the CPA cocktail of the first loading step can be between about 5 weight percent and about 15 weight percent, or between about 8 weight percent and about 12 weight percent, or about 11 weight percent. In some embodiments, the amount of CPA in the CPA cocktail of the second loading step can be between about 15 weight percent and about 30 weight percent, or between about 20 weight percent and about 25 weight percent, or about 22 weight percent. In some embodiments, the amount of CPA in the CPA cocktail of the third loading step can be between about 35 weight percent and about 50 weight percent, or between about 40 weight percent and about 48 weight percent, or about 44 weight percent.

In some embodiments, the amount of CPA in the CPA cocktail in the first step can be about ¼ of the weight percent of the CPA in the third and/or final step. In some embodiments, the amount of CPA in the CPA cocktail in the second step can be about ½ of the weight percent of the CPA in the third and/or final step. It will be understood that other gradual increases in CPA concentrations can be used in each successive step.

In some embodiments, the CPA concentration may be increased in a gradual and continuous manner by flowing in or pumping in the CPA into a suspension containing the biological material until the concentration of the CPA in the CPA cocktail is at about the vitrification CPA concentration. In some embodiments, to achieve the gradual increase of CPA concentration over time, the full strength CPA solution can be added at a desired flow rate using a syringe, peristaltic, or similar pump to the initial culture media to achieve a continuous, ramped increase of the biological material to the vitrification CPA concentration as shown, for example, in FIG. 14. The duration of the continuous addition of the CPAs can vary and can be, for example, at least about 10 minutes, or at least about 30 minutes, or at least about 60 minutes, or at least about 4 hours.

In some embodiments, the vitrification CPA concentration in the CPA cocktail can be determined and then the concentrations of the CPA in the CPA cocktails in the preceding steps can be determined that maintain the integrity, e.g. shrinking/swelling, of the biological material.

In some embodiments, the stepwise CPA addition or the continuous CPA addition can be used to minimize osmotic damage. Gradual addition with time allotted for equilibration can maintain the biological materials without extreme swelling and/or shrinking volumes. Addition of CPA in a gradual manner can allow the biological material to adjust to the higher concentrations of the CPA in stages without excessive shrinkage or swelling. Without being bound by any theory, it is thought that the “shrink” occurs due to high water permeability upon initial CPA exposure, driving water efflux from the islets. This shrink is followed by a “swell” as permeable CPA diffuses across the cell membrane and water slowly re-enters the islets. After some time, the cells can swell back to about the natural cellular volume with equilibration. Allowing time for the biological material to equilibrate can minimize the amount of excessive shrinkage or swelling.

In some embodiments, the shrinking and swelling of the biological material during the loading and the unloading of the CPA cocktail is between about 50% and about 160% of the initial volume of the biological material, or between about 60% and about 155%, or between about 70% and about 140% of the initial volume of the biological material. In one embodiment, the biological material can shrink to about 60% of the initial volume and swell to about 155% of the initial volume during the loading and the unloading of the CPA cocktail. In one embodiment, the biological material can shrink to about 70% of the initial volume and swell to about 120% of the initial volume during the loading and the unloading of the CPA cocktail.

In some embodiments, the CPA cocktail that would avoid ice formation while minimizing toxicity can be identified. In one embodiment, a correlation between CPA concentration and CWR, the minimal CPA concentration in the interior of an islets can be determined by examining the biophysical parameters of the biological material. In one embodiment, the amount of CPA in the CPA cocktail can be about 42 to about 44 wt % to avoid devitrification (ice formation) during rewarming. In one embodiment, the CPA cocktail can include two or more cryoprotective agents. In one embodiment, the CPA cocktail can include about 22% by weight of EG and about 22% by weight of DMSO. Other combinations of CPAs and weight percentages may be used and are within the scope of this description.

In some embodiments, two CPAs, e.g. EG and DMSO, may be used at about a 1:1 ratio, or about 1:2 ratio, or about 1:5 ratio, or about 1:10 ratio or about 2:1 ratio, or about 5:1 ratio, or about 10:1 ratio in the CPA cocktail. Other ratios and combinations of CPAs used in the CPA cocktail are also in the scope of this description.

The method can include unloading the CPAs in the rewarmed biological material. Unloading as referred to herein relates to removal of CPAs from the biological material after rewarming from a cryogenic temperature. In some embodiments, unloading of the CPAs from the rewarmed biological material can be performed by gradually decreasing the CPA concentration after rewarming of the vitrified biological material. In some embodiments, the unloading may be performed in multi-steps. In some embodiments, the unloading may be performed by gradually flowing in or pumping in a diluent or buffer to slowly reduce the concentration of the CPAs in the CPA cocktail.

In some embodiments, the unloading step can include the addition of a non-penetrating CPA, e.g. sucrose. In the unloading solutions, sucrose may be added to raise the solution osmolality and avoid osmotic shock. The concentration of the sucrose added to the VR biological material may be gradually increased as the unloading proceeds. The concentration of the sucrose can vary and all are within the scope of this description. In some embodiments, the concentration of the sucrose may be at least about 2% by weight, or at least about 3% by weight, or at least about 4% by weight or at least about 5% by weight at the beginning of unloading. In some embodiments, the sucrose may be increased to at least about 6% by weight, or at least about 7% by weight, or at least about 8% by weight, or at least about 9% by weight when the unloading is completed. In one embodiment, the concentration of the sucrose may be about 5% by weight at the beginning of unloading and may be increased to about 8.75% when the unloading is completed. In some embodiments, the sucrose can be added to a diluent or buffer.

In some embodiments, a stepwise unloading can be performed after rewarming the vitrified biological material to minimize osmotic damage. Stepwise removal can maintain the biological materials without extreme swelling and/or shrinking volumes. Removal of CPA in a stepwise manner can allow the biological material to adjust to the lower concentrations of the CPA without excessive shrinkage or swelling. Without being bound by any theory, it is thought that while the above describes the shrink-swell during loading, the inverse occurs during unloading, which is a swell followed by a shrink. During unloading, the cells can swell and then shrink back to the natural cellular volume after equilibration. Once the sample has equilibrated, the sample can then be placed in a CPA cocktail with a lower concentration of CPA. This allows for gradual decrease in CPA in the sample without causing excessive shrinkage or swelling.

In some embodiments, the amount of CPA in the CPA cocktail of the first unloading step can be between about 15 weight percent and about 30 weight percent, or between about 20 weight percent and about 25 weight percent, or about 22 weight percent. In some embodiments, the amount of CPA in the CPA cocktail of the second unloading step can be between about 5 weight percent and about 15 weight percent or between about 8 weight percent and about 12 weight percent, or about 11 weight percent. In some embodiments, the amount of CPA in the CPA cocktail of the third unloading step can be between about 3 weight percent and about 12 weight percent, or between about 5 weight percent and about 10 weight percent, or about 6 weight percent. In some embodiments, the first unloading step can include a non-penetrating cryoprotectant, e.g. sucrose, at a concentration of between about 3% and about 7% or between about 4% and about 6% by weight or about 5%. In some embodiments, the second unloading step can include a non-penetrating cryoprotectant, e.g. sucrose, at a concentration of between about 6% and about 9% or between about 7% and about 8% by weight or about 7.5%. In some embodiments, the third unloading step can include a non-penetrating cryoprotectant, e.g. sucrose, at a concentration of between about 7% and about 10% or between about 8% and about 9% by weight or about 9.5%.

In some embodiments, the CPA concentration may be decreased in a gradual and continuous manner by a flowing in a diluent into a suspension containing the VR biological material until the CPA concentration surrounding the VR biological material is negligible. The unloading may be completed by placing the VR biological material in a CPA free buffer. In some embodiments, to achieve the gradual decrease of CPA concentration over time, the diluent or buffer can be added at a desired flow rate using a syringe, peristaltic, or similar pump to the VR biological material to achieve a continuous, decrease of exposure of the VR biological material to the CPAs. The duration of the continuous decrease of the CPAs can vary and can be, for example, at least about 10 minutes, or at least about 30 minutes, or at least about 60 minutes, or at least about 4 hours.

In some embodiments, the biological material may shrink in volume in each step, during loading or unloading, by no more than about 65% of the natural cell volume, or no more than about 60%, or no more than about 50%, or no more than about 40%, or no more than about 30%, or no more than about 20%, or no more than about 10% of the natural cell volume.

In some embodiments, the biological material may swell in volume, during loading or unloading, be within about 165%, of the natural biological material volume, or within about 160%, or within about 150%, or within about 140%, or within about 130%, or within about 120%, or within about 110% of the natural biological material volume. In one embodiment, the volume of the cell can shrink by no more than about 60% and the volume may swell and be within about 155% of the natural cell volume during loading and unloading.

The duration of each of the steps during loading and unloading of the CPA cocktail can vary and all are within the scope of this description. Duration of the step can be sufficient to allow for the cells to equilibrate and return to a natural cell volume after shrinking/swelling during loading or swelling/shrinking during unloading. In some embodiments, the duration of each step can be between about one minute and about 30 minutes, or between about 5 minutes and about 20 minutes, or about 10 minutes. In one embodiment, the duration of each of the loading steps can be about 10 minutes. In one embodiment, the duration of each of the unloading steps can be between about 5 minutes and about 10 minutes.

The temperature at which the loading and unloading steps are conducted can vary. A higher temperature may be used at lower concentrations of CPA and a lower temperature can be used at higher concentrations of CPA. Without being bound by any theory, it is thought that as the concentration of the CPA increases the temperature at which the biological material is exposed to the CPA should be decreased to reduce or minimize the chemical toxicity to the biological material. In some exemplary embodiments, room temperature or a temperature of about 21° C. can be used when the CPA concentration is below about 2M or below about 1.5M. In some exemplary embodiments, a temperature of about 4° C. or lower may be used when the CPA concentration is at or above about 1.5M or at or above about 2M. These temperatures and concentrations are exemplary and may change and may be dependent on the specific biological material, the CPAs in the CPA cocktail and the like.

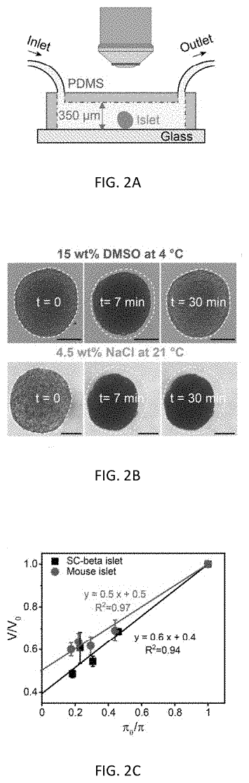

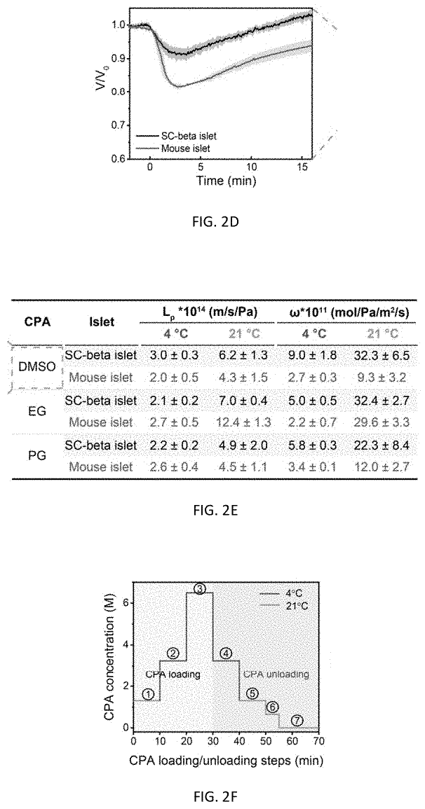

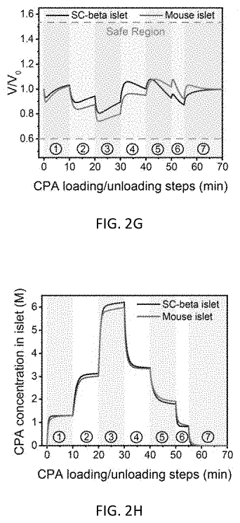

In one exemplary embodiment, the CPA loading can include 3 loading steps. Each of the steps can have a duration of about 10 minutes. In one embodiment, the first loading step can include CPA at about 1.3 M in the CPA cocktail and the step can be conducted at about 21° C. In one embodiment, the second loading step can include CPA at about 3.2M in the CPA cocktail and the step can be conducted at about 4° C. In one embodiment, the third loading step can include CPA at about 6.5M in the CPA cocktail and the step can be conducted at about 4° C. This can result in a CPA concentration inside the islets of about 6.2M. (See FIGS. 2F-2H).

In one exemplary embodiment, the unloading of the CPA from the biological material after rewarming can include 3 unloading steps.

The present description can further include methods that use the cryomesh described herein after loading the biological material with the CPA(s). The method can include the use of a cryomesh for vitrification and rewarming of the biological specimen. The method can maintain high cooling and/or rewarming rates. In some embodiments, the cryopreservation method can include cooling the biomaterial specimen. The method can include transferring the biomaterials that have been loaded with CPA in a CPA cocktail to the cryomesh. The biomaterials in the CPA cocktail can be transferred onto the mesh in a variety of methods. In some embodiments, a volume of CPA cocktail with the biological specimen may be placed on the cryomesh. The placement of the biomaterials and the CPA cocktail onto the mesh can result in some or most of the CPA cocktail being removed from the biomaterials by drainage of the CPA cocktail through the openings in the mesh or through another porous surface. In some embodiments, a wicking material and/or an external vacuum can be used to remove or wick away the CPA cocktail around the biological sample. The cryomesh with the biological specimen can then be submerged into a cryogenic coolant to rapidly cool the specimen. Advantageously, wicking the CPA cocktail around the biological sample can minimize the thermal mass of the specimen being cryopreserved and effectively increase the cooling rates achieved.

In one exemplary embodiment, as shown in FIG. 1E, biological specimen 138 is combined with a CPA cocktail 134 in vessel 132. In some embodiments, the method can include combining the biomaterials with a CPA cocktail in vessel, e.g. a test tube, a pan and the like. The biomaterials, the CPA cocktails are described in more detail herein. A volume of droplet 140 that includes CPA cocktail 134 and specimen 138 is transferred onto mesh 120 of cryomesh 100. Some of CPA cocktail 134 drains through gaps of mesh 120. In some embodiments, wicking material 130 may be placed adjacent to mesh 120 to wick CPA cocktail 134 away from biological specimen 138. It is advantageous to remove all or most of the CPA cocktail 134 from being in contact with biological specimen 138 prior to cooling. In some embodiments, an external vacuum may also be used to remove all or most of the CPA cocktail 134 from biological specimen 138.

In some embodiments, the wicking can remove all of the CPA cocktail around the biological sample; or greater than about 90% of the CPA cocktail; or greater than about 80% of the CPA cocktail; or greater than about 50% of the CPA cocktail around biological sample.

In some embodiments, the wicking material may be fibrous. In some embodiments, the wicking material may be placed on, placed below and/or be resting on/around the mesh to advantageously wick any moisture that may be present in the sample. The fibrous wicking material can be, for example, a fibrous tissue. The thickness of the fibrous wicking material can vary. The fibrous wicking material can have a thickness of at least about 0.1 mm. In some embodiments, the thickness of the fibrous wicking material is between about 0.1 mm and about 2 mm. Thickness outside of this range are also within the scope of this disclosure.

The method can further include placing mesh 120 with specimen 138 into cryogenic coolant 152 in cryogenic container 150. Cryogenic coolant 152 can include, for example, liquid nitrogen. Cryogenic coolant 152 may also include slush nitrogen. Other cryogenic coolants such as ethanol, methanol, FC 770 oil (3M) may also be used and all are within the scope of this description.

In some embodiments, the method can include cooling for vitrification of the islets. The cryomesh with the islets can be quickly plunged into a cryogenic coolant, e.g. liquid nitrogen. The cryogenic coolant can be liquid nitrogen, slush nitrogen and the like. At this stage the islets can be stored in the cryogenic coolant until future use or transferred to another means of maintaining them at cryogenic storage temperatures.

In some embodiments, the use of cryomesh in the cryopreservation methods can increase the cooling and rewarming rates and/or increase the throughput over prior art methods. In some embodiments, the cooling rates can be greater than about 25,000° C./min; or greater than about 30,000° C./min; or greater than about greater than about 40,000° C./min; or greater than about 50,000° C./min; or greater than about 60,000° C./min; or greater than about 100,000° C./min; or greater than about 500,000° C./min; or greater than about 700,000° C./min; or greater than about 1,000,000° C./min.

The vitrified islets may be stored at cryogenic temperatures for an indefinite period of time and until desired future use. In some embodiments, the islets may be stored for more than a day; or more than a week; or more than a month; or more than 6 months; or more than a year; or more than 5 years.

In some embodiments, the method can further include rewarming the cryopreserved islets. A variety of rewarming methods can be used to rewarm the cryopreserved biological sample and all are within the scope of this description. In some embodiments, the biological sample may be rewarmed by convective methods. Convective methods can include, for example, incubation of the cryopreserved sample in an unloading solution, plunging the cryopreserved sample in an unloading solution, plunging the cryopreserved sample in an unloading solution and agitating the sample or solution, and the like.

In some embodiments, the warming rates can be greater than about 100,000° C./min; or greater than about 150,000° C./min; or greater than about 200,000° C./min; or greater than about 300,000° C./min; or greater than about 400,000° C./min; or greater than about 1,000,000° C./min; or greater than about 2,000,000° C./min; or greater than about 3,000,000° C./min; or greater than about 4,000,000° C./min; or greater than about 5,000,000° C./min.

The vitrified biological material can be rewarmed to a temperature that minimizes the chemical toxicity of the CPAs present in the vitrified biological material prior to initiating the unloading of the CPAs. The rewarmed biological material may be immediately subjected to unloading of the CPAs to minimize chemical toxicity. In some embodiments, the vitrified biological material may be rewarmed to a temperature below about 10° C., or below about 4° C. or below about 0° C., or below about −5° C., or below about −10° C. In one embodiment, the vitrified biological material may be rewarmed to a temperature between about −5° C. and about 5° C.

A variety of biological materials can be cryopreserved using the methods described herein and all are within the scope of this description. In some embodiments, the methods described herein can be effective for cryopreserving freshly isolated mouse, porcine, human pancreatic islets and SC-derived human islets. Islets isolated from other sources are also within the scope of this description. In one embodiment, SC-beta islets can provide a promising source of human cells for beta cell replacement therapy, demonstrating glucose-responsive insulin secretion in vitro and long-term glycemic control in diabetic mouse models. The cryopreservation method described herein can provide a high viability and in vitro and in vivo functionality of SC-beta islets, even after 9 months of storage in liquid nitrogen. Long-term storage can allow immune manipulation of prospective recipients, enable comprehensive islet quality control prior to transplantation, and increase cost efficiency by allowing large batches to be cryopreserved in functional single-patient units.

The vitrified and rewarmed biological material may be analyzed to determine the recovery, viability and the functional characteristics. Recovery as used herein is the percent of islets, live and dead, retrieved in the VR biological material relative to the starting quantity. Viability is the number or percentage of viable islets in the recovered VR biological material relative to a fresh control.

The viability of the VR biological material can vary and can be dependent on the specific biological material, the specific CPAs used, the concentration of CPAs used, the CCR and CWR. In some embodiments, the viability of the VR islets is at least about 75%, or at least about 80%, or at least about 85%, or at least about 90%, or at least about 95% relative to the control. “Control” sample or material as used herein is an islet sample that has not been through vitrification and rewarming. The high viability rates are advantageous for use in therapeutic uses. In some embodiments, the high viability remained unchanged for at least 9 months of cryogenic storage.

The VR biological materials may also be analyzed for functional characteristics. The specific characteristics analyzed can be dependent on the nature of the biological material. In some embodiments, the VR biological materials can be allowed to recover before analysis for functional characteristics and/or use for therapeutic uses. The VR biological materials may be allowed to recover for at least about 30 minutes, or at least about one hour, or at least about 2 hours, or at least about 3 hours prior to characterization or use. In one embodiment, the VR biological materials are allowed to recover for about 3 hours prior to characterization or use. Recovery can be performed by incubating the VR biological materials under ex vivo conditions that mimic their normal physiological state, allowing for improved functional characteristics as compared to VR biological materials treated without a recovery period. Recovery can be performed in a dynamic culture flask maintained at 70 RPM. In some embodiments, dynamic culture can be maintained at least about at 10 RPM, at or least about at 20 RPM, or at least about at 50 RPM, or at least about at 70 RPM. In one embodiment, recovery of the VR biological materials may be conducted under dynamic culture maintained at 70 RPM.

In some embodiments, the VR islets can have normal macroscopic, microscopic, and ultrastructural morphology as demonstrated in the Examples below. In some embodiments, mitochondrial membrane potential and ATP levels may be slightly reduced, but all other measures of cellular respiration, including oxygen consumption rate (OCR) to produce ATP, may be unchanged. See Examples below.

In some embodiments, the mitochondrial membrane potential of the VR islets is within at least about 60% of the control islets, or within at least about 70% of the control islets, or within about 80% of the control islets, or within about 90% of the control islets.

In some embodiments, the ATP levels of the VR islets is within at least about 60% of the control islets, or within at least about 70% of the control islets, or within about 80% of the control islets, or within about 90% of the control islets.

In some embodiments, the OCR of the VR islets is within at least about 70% of the control islets, or within at least about 80% of the control islets, or within about 90% of the control islets, or within about 95% of the control islets.

In some embodiments, VR islets may have normal glucose-stimulated insulin secretion function in vitro and in vivo. In some embodiments, the glucose-stimulated insulin secretion of the VR islets indicates transplant success within at least about 90% of the transplant cases, or within at least about 95% of the transplant cases, or within at least about 99% of the transplant cases.

In the methods described herein, porcine and SC-beta islets can generate insulin in xenotransplant models, and mouse islets tested in a marginal mass syngeneic transplant model can cure diabetes in at least about 70% of the recipients, or in at least about 80% of the recipients, or in at least about 90% of the recipients. In one embodiment, the syngeneic transplant can cure diabetes in about 92% of recipients within 24-48 hours following transplant. Excellent glycemic control can be seen for at least about 150 days or at least about 200 days, or at least about 300 days.

In one embodiment, the CPA formulation can include a cocktail of EG and DMSO. The CPA loading and unloading design may have minimal osmotic and accumulative chemical damages using the measured islet biophysical parameters and model-derived CPA loading protocol. In one embodiment, the viability of SC-beta islets remained at 96.4% after loading and unloading of 22 wt % EG+22 wt % DMSO. In some embodiments, high viabilities post VR for porcine (87.2%) and human islets (87.4%) can also be seen.

The methods described herein can be scalable for generating large amounts of VR islets with high recovery, viability and functionality. In one embodiment, the methods described herein can be used to process at least 2,500 islets with >95% islets recovery at >89% post-thaw viability. In some embodiments, the method can be scaled to increase the number of VR islets per batch by increasing the length and width of the cryomesh. In some embodiments, the method can be scaled to increase the number of VR islets per run by stacking cryomesh as illustrated, for example, in FIGS. 14A-14D. In some embodiments, the method can produce at least about 10,000 islets per batch, or at least about 30,000 islets per batch, or at least about 50,000 islets per batch, or at least about 75,000 islets per batch, or at least about 100,000 islets per batch, or at least about 200,000 islets per batch, or at least about 300,000 islets per batch, or at least about 500,000 islets per batch.

The method described herein can be conveniently scaled up using larger cryomesh sizes and alternative form factors such as mesh stacking. The residual CPA maintaining contact between the islets and cryomesh due to surface tension, allows the vitrified islets to remain adhered to the cryomesh in cryogenic storage. UV radiation has been shown to successfully kill micro-organisms that can otherwise survive in liquid nitrogen, and can thus ensure sterility of the samples during cryogenic storage.

In some embodiments, the present description can include a method for banking a supply of donor biological material. The method can include loading the biological material with a CPA cocktail for vitrification as described herein. The method can include transferring the CPA loaded biological material onto a cryomesh and removing the CPA cocktail surrounding the CPA loaded biological material prior to cooling the CPA loaded biological material. The biological material can be vitrified by cooling the CPA loaded biological material on the cryomesh to form a vitrified biological material. The method can include rewarming the vitrified biological material, when desired, and unloading the CPA from within the rewarmed biological material as described herein to form a vitrified and rewarmed (VR) biological material.

In some embodiments, the present description can include a method of therapeutic transplantation of a biological material comprising transplanting the VR biological material into a patient. The therapeutic transplantation can be therapeutic for patients with, for example, diabetes. The biological material can include VR islets derived from one or more donors. In some embodiments, the VR islets are derived from 2 or more donors, or 3 or more donors.

The VR islets can have a viability of at least 80 percent, or at least 85%, or at least 90% or at least 95% relative to the control. The biological material can include at least about 10,000 VR islets from one donor, or at least about 50,000 VR islets from one donor, or at least about 75,000 VR islets from one donor, or at least about 100,000 VR islets from one donor, or at least about 200,000 VR islets from one donor, or at least about 500,000 VR islets from one donor. The biological material can include at least about 10,000 VR islets from two or more donors, or at least about 50,000 VR islets from two or more donors, or at least about 75,000 VR islets from two or more donors, or at least about 100,000 VR islets from two or more donors, or at least about 200,000 VR islets from two or more donors, or at least about 500,000 VR islets from two or more donors.

The biological material for transplantation can be pooled from two or more donors. The biological material can be pooled prior to cryopreservation and/or after rewarming. The biological material to be transplanted to the patient can include at least about 100,000 VR islets, or at least about 200,000 VR islets, or at least about 300,000 VR islets, or at least about 400,000 VR islets, or at least about 500,000 VR islets, or at least about 700,000 VR islets, or at least about one million VR islets.

The biological material for transplantation can be SC-derived islets which can be produced in large batches and cryopreserved as VR islets for therapeutic use as single-patient doses. The biological material to be transplanted to the patient can include at least about 100,000 VR islets, or at least about 200,000 VR islets, or at least about 300,000 VR islets, or at least about 400,000 VR islets, or at least about 500,000 VR islets, or at least about 700,000 VR islets, or at least about one million VR islets. Production and subsequent storage of large batches intended for single therapeutic administration can enable comprehensive islet quality control, lot tracking, and assessment prior to transplantation.

SC-derived islets can provide an unlimited islet supply. However, heterogeneity in endocrine cell composition and variability in function can lead to significant batch-to-batch variability, which can require extensive pre-transplant validation of each lot, during which time SC-islets deteriorate in culture. Further, biologic potency testing can be a critical element of the regulatory framework for processing human-derived cell and tissue products. Under the FDA final rule, measurement of potency is a requirement for allogeneic islet products, and establishing the accuracy, sensitivity, specificity, and reproducibility of the potency assay must demonstrate lot-to-lot consistency and stability of the product. One of the main challenges in islet transplantation has been the lack of a rapid and definitive test of biological potency. The gold standard for islet transplantation is the cure of diabetes in a nude mouse bioassay (NMB) system, which takes weeks to produce accurate results. More rapid in vitro assays do not always predict function in vivo. With VR islets, more accurate testing methods (such as the NMB) can be used to define the relative potency (and stability of potency) of islets and SC-derived islet products.

In some embodiments, the present description can include a vitrified and rewarmed biological material. The biological material can include, for example, islets. The islets can have a recovery, viability and functionality as described herein. In some embodiments, the VR biological material can have a cellular respiration, oxygen consumption rate to produce ATP that are substantially similar (within 90% relative) to a control. In some embodiments, the islets may have been cooled at a rate greater than about 50,000° C./min and rewarmed at a rate greater than about 200,000° C./min. The VR biological material can include VR islets greater than about 2500 VR islets. The VR biological material can include VR islets greater than about 10,000 VR islets from one donor. The VR biological material can include VR islets greater than about 50,000 VR islets from one donor. The VR biological material can include VR islets greater than about 100,000 VR islets from one donor.

Advantageously, the scalability of the method can improve efficacy through high-dose pooled donor islet transplants; better opportunity for quality control and assessment; better patient preparation by converting an unplanned operation into a planned event; decreased risk by using a single islet infusion rather than repeated infusions; improved opportunity for HLA matching of donor and recipient by selecting from a bank of available options rather than using the next donor in line; facilitated tolerance induction protocols that require recipient preconditioning before transplants, such as tolerance achieved by apoptotic donor leukocytes infusion or mixed chimerism with islets and donor-derived hematopoietic stem cell transplant; and improved organ utilization by promoting islet isolation and banking from all appropriate donors rather than only “optimal” donors that have been historically selected to maximize yields in hopes of single-donor transplants.

### EXAMPLES

### Example—Cryopreservation of Pancreatic Islets

Methods

Islet Isolation Methods

Institutional IACUC committees approved all animal studies. Mouse islets were isolated from C57BL/6 female retired breeders (Charles River Laboratories, Wilmington, Mass.) by collagenase (Clzyme RI, VitaCyte, Indianapolis, Ind.) digestion and Histopaque (Sigma) density gradient enrichment, as previously described in Melli K., et al. p. 2590-2600 (2009). Islets were then handpicked and cultured in a bioreactor (ABLE Biott reactor; Cat #BWV-503A) in S3 media for 16 hours before use. Human islets were purchased from a commercial vendor (Prodo Laboratories, Aliso Viejo, Calif.) and cultured in PIM(S) Complete media (Prodo Laboratories) for up to 7 days prior to use. Porcine islets were isolated from adult Landrace pigs as described in Anazawa, T., et al (2010).

Stem Cell-Derived Islets Differentiation

HUES8 cells were cultured as spheroids in 500 mL spinner flasks (Corning, 3153), as previously described in Pagliuca, F. W., et al. p. 428-439 (2014). Suspension cultures were established by seeding 150 million cells (5×10⁵ cells/mL) in mTeSR1 media (STEMCELL Technologies, 85850) with 10 μM Y27632 (R&D Systems, 1254), and maintained at 70 RPM inside the humidified incubator at 37° C., 5% CO2, and 100% humidity. Media was changed at 48 hours to mTeSR1 without Y27632. Cells were passaged every 72 hours by dispersing to single cells using Accutase (Sigma, A6964) with mechanical disruption and resuspended in fresh mTeSR1 with Y27632.

SC-beta differentiations were initiated after 3 days of stem cell spheroid formation and expansion in mTeSR1 media inside a spinner flask, as previously described in Pagliuca, F. W. et al. (2014) and Veres, A., et al. (2019). Clusters were allowed to settle by gravity, and the media was replaced with protocol-specific media including appropriate growth factors. Cell differentiation was directed sequentially to definitive endoderm (DE), primitive gut tube (PGT), pancreatic progenitor 1 (PP-1), pancreatic progenitor 2 (PP-2), endocrine progenitors (EN), and finally into β-cells (SC-beta). Basal media types (S1, S2, S3, and BE5) were supplemented with inductive signals, as described below.

S1 media was comprised of 1 L MCDB 131 (Life Technologies, 10372019) supplemented with 0.44 g glucose, 2.46 g sodium bicarbonate, 20 g fatty-acid-free bovine serum albumin (FAF-BSA, Proliant Biologicals, 68700), 20 μL ITS-X (Life Technologies, 51500-056), 10 mL, Glutagro (Corning, 25-015-C1), 44 mg ascorbic acid, and 10 mL penicillin/streptomycin (P/S) solution (30-001-C1). S2 media: 1 L MCDB 131 supplemented with 0.44 g glucose, 1.23 g sodium bicarbonate, 20 g FAF-BSA, 20 μL ITS-X, 10 mL Glutagro, 44 mg ascorbic acid, and 10 mL P/S. S3 media: 1 L MCDB 131 supplemented with 0.44 g glucose, 1.23 g sodium bicarbonate, 20 g FAF-BSA, 5 mL ITS-X, 10 mL Glutagro, 44 mg ascorbic acid, and 10 mL P/S. BE5 media: 1 L MCDB 131 supplemented with 3.6 g glucose, 1.754 g sodium bicarbonate, 20 g FAF-BSA, 5 mL ITS-X, 10 mL Glutagro, 44 mg ascorbic acid, 10 mL P/S, and 4000 units heparin (MilliporeSigma, H3149).

Directed differentiation of pluripotent stem cells to SC-beta cells was performed by changing media within the spinner flask and supplementation with small molecules and growth factors specific to the differentiation stage. Media changes are as follows: Day 1: S1 media+100 ng/mL Activin A+3 μM CHIR99021; Day 2: S1 media+100 ng/mL Activin A; Day 4: S2 media+50 ng/mL KGF; Day 6: S3 media+50 ng/mL KGF+250 nM Sant-1+500 nM PDBu+200 nM LDN 193189+2 μM RA+10 μM Y27632; Day 7: S3 media+50 ng/mL KGF+250 nM Sant-1+500 nM PDBu+2 μM RA+10 μM Y27632; Days 8, 10, 12: S3 media+50 ng/mL KGF+250 nM Sant-1+100 nM RA+10 μM Y27632+5 ng/mL Activin A; Days 13+15: BE5 media+250 nM Sant-1+20 ng/mL betacellulin+1 μM XXI+10 μM ALK5i+1 μM T3+100 nM RA; Days 17+19: 20 ng/mL betacellulin+1 μM XXI+10 μM ALK5i+1 μM T3+25 nM RA; Days 20-26: S3 media only.

KGF (cat #100-19) was purchased from Peprotech. All other factors were purchased from R&D Systems with the following catalog numbers: Activin A (338-AC), CHIR (4423), SANT-1 (1974), PDBu (4153), RA, Retinoic Acid (0695), LDN, LDN193189 (6053), Y27632 (1254), Betacellulin (261-CE), ALK5i (3742), T3, L-3,3′,5-Triiodothyronine (5552), and XXI, γ-secretase inhibitor XXI (6476).

Flow Cytometry for SC-Beta Characterization

Spheroids were dispersed into single cells by incubation in TrypLE Express at 37° C. for 10 minutes and mechanically disrupted. Cells were quenched with S3 media, washed with PBS, fixed in 4% PFA for 60 min, and stored at 4° C. in PBS until further use. Before staining, cells were filtered through a 40 μm cell strainer, permeabilized in blocking buffer (lx PBS, 0.1% Triton X-100, 5% donkey serum) at room temperature for 40 minutes, and washed three times in PBST (0.1% Triton X-100). Cells were then incubated with primary antibodies in the blocking buffer for 1 hour at room temperature, washed 3 times with PBST, and incubated with secondary antibodies in blocking buffer for 1 hour at room temp. After labeling, cells were washed 3 times, resuspended in PBST at a concentration of 1×10⁶ cells/mL, and analyzed using an Attune flow cytometer (ThermoFisher, Waltham, Mass.). Data were acquired and analyzed using NXT v. 3.1 (ThermoFisher) and FlowJo v.10 (FlowJo, Ashland, Oreg.) software.

The following primary antibodies were used for flow cytometry: goat anti-PDX1 (R&D Systems, AF2419), mouse anti-NKX6.1 (DSHB, F55A12), rabbit anti-chromogranin (Abcam, ab15160), rat anti-c-peptide (DSHB, GN-ID4), and mouse anti-glucagon (Sigma, G2654). Secondary antibodies used for flow cytometry were donkey anti-goat Alexa Fluor 488 (Invitrogen, A-11055, 1:1,000), donkey anti-rabbit Alexa Fluor 488 (Invitrogen, A-21206, 1:1,000), donkey anti-rat Alexa Fluor 488 (Invitrogen, A-21208, 1:1,000), and donkey-anti-mouse-Alexa Fluor 647 (Invitrogen, A-31571, 1:1,000).

Measurement of Islet Biophysical Parameters

The microfluidic devices used to measure islet biophysical parameters were fabricated using soft lithography procedures. A 350 μm thick SUEX epoxy film sheet (DJ MicroLaminates, Sudbury, Mass.) was bonded to the surface of a silicon wafer using hot-roll lamination. The sheet was then photopatterned with the channel geometry, generating a mold for creating subsequent devices. After exposing the wafer to silane vapors in a desiccator for 20 minutes, a 10:1 by weight mixture of polydimethylsiloxane (PDMS) prepolymer and curing agent (SYLGARD 184 Silicone Elastomer Kit, Dow Corning Corporation, US) was poured over the mold and incubated overnight at 70° C. for cross-linking. PDMS devices were cut out from the wafer and holes for tubing connections were punched using a 1.5 mm OD biopsy punch (Integra LifeSciences, Princeton, N.J.). The PDMS device and glass slide were sealed after activating the bonding surfaces using oxygen plasma (Harrick Plasma, Ithaca, N.Y.) at 18 W for 1 minute and then stored at 70° C. for 2 hours. To render the PDMS channel walls hydrophilic, the device was treated with oxygen plasma for 15 minutes. The devices were soaked in water until usage to maintain channel hydrophilicity. The devices were mounted on the top of an inverted microscope for use, and the channels were flushed with the culture medium.

Handpicked islets were introduced into the device in retrograde fashion through the outlet hole. After initiating antegrade flow using islet media, CPA/salt solution of the desired concentration was loaded via a syringe pump. For CPAs, 15 wt % EG, PG, and DMSO prepared in RPMI were used. For salt solutions, 1.8%, 2.7%, 3.6% and 4.5% sodium chloride prepared in deionized water were used. Once the islet settled on the channel floor, the CPA/salt solution flowrate was gradually increased while minimizing movement and rotation of the islet. The solution flow was stopped after at least 5 device volumes of solution had been pumped through the device. For experiments performed at 4° C., the entire experiments were performed inside a cold room maintained at the same temperature. Islet cross-sectional area changes were recorded and were analyzed using MATLAB to estimate islet spherical volume changes.

Fitting of Lₚ and ω

The water and CPA transport phenomena in the islets are described by the Kedem-Katchalsky (K-K) equation. The assumptions include that the cell membrane has constant lumped properties and that irreversible thermodynamics can be applied to study the nonelectrolyte solute (CPA) and solvent (water) transports. A two-parameter model (2P) utilizing Lp (hydraulic conductivity, also called water permeability) and ω (CPA permeability) was applied to fit the experimental shrink-swell data (Kleinhans, F. W. (1998)). The hydraulic conductivity is a measure of the mechanical filtration capacity of the membrane or the velocity of water moving through the membrane per unit pressure difference, and the CPA permeability is a measure of the permeable solute transport across the membrane. Both parameters are temperature dependent, following the Arrhenius relationship. The osmotic inactive cell volume, Vb, was determined from the Boyle-van′ t Hoff relationship. The Boyle-van′t Hoff equation correlates the cell equilibrium volume with the osmolality of non-permeating solution as below:

$$\frac{V}{{V}_{0}} = ( 1 - {V}_{b} ) \frac{{π}_{0}}{π} + {V}_{b} \qquad ( 1 )$$

where V is the cell equilibrium volume, V₀ is the isotonic cell volume, V_{b} is the osmotically-inactive volume fraction of the cell, π₀ is the isotonic osmolality, π is the osmolality of the non-permeating solution.

MATLAB was used to fit the Lₚ and ω of the mouse and SC-beta islets from the experimental shrink and swell curves.

Modeling of islet volume change and intracellular CPA is based on the 2-parameter “uncoupled” model suggested by Kleinhans, as follows:

$$\frac{dV}{dt} = - {L}_{P} ART ( {C}_{s}^{e} - {C}_{s}^{i} + {C}_{C}^{e} - {C}_{C}^{i} ) \qquad ( 2 )$$

$$\frac{{dn}_{c}}{dt} = ω RTA ( {C}_{C}^{e} - {C}_{C}^{i} ) \qquad ( 3 )$$

Where V and A are the islet volume and surface area, respectively, n_{c} is the number of moles of CPA inside the islet, R is gas constant, T is the absolute temperature, Lₚ and co are the hydraulic conductivity and membrane permeability to CPA, respectively and C is the molality. The superscripts i and e denote intracellular and extracellular, respectively. The subscripts C and S denote permeating CPA and non-permeating solutes, respectively.

Chemical Toxicity Cost Function

To minimize the chemical toxicity, the toxicity cost function developed by Benson et al. was adapted to evaluate the effect of step duration of CPA loading and unloading. Briefly, cumulative temperature and concentration-dependent chemical toxicity can be quantitatively described by the Jₜₒₓ, as shown below.

$$\{ \begin{matrix}k = β \cdot {C}_{CPA}^{α} \\ \frac{dN}{dt} = - k \cdot N \\ {J}_{t o x} = {\int }_{0}^{{t}_{f}} k dt = {\int }_{0}^{{t}_{f}} β \cdot {C}_{CPA}^{α} d t \\ \frac{N}{{N}_{0}} = exp ( - {J}_{tox} )\end{matrix} \qquad ( 4 )$$

where k is toxicity rate, α and β are two constants that depend on the type of CPA, C_{CPA} is the intracellular CPA concentration, N is the viability after exposure, N₀ is the initial viability, t_{f} is the duration of CPA exposure, and Jₜₒₓ is the toxicity cost function.

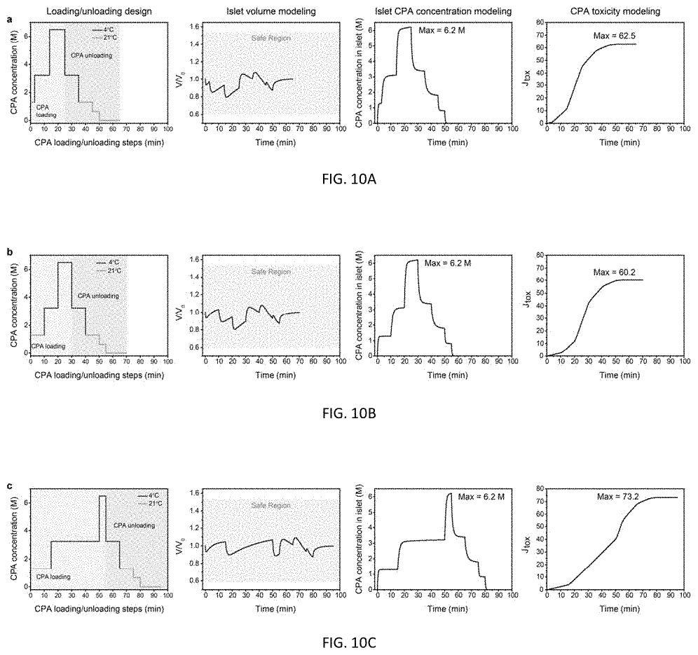

Using the SC-beta islets as the model system, short, medium, and long CPA loading step durations were tested that achieve the same final CPA concentration (6.2M) inside the islets (FIGS. 10A-10C). For short CPA loading duration, 3 min in 20% CPA at 21° C., 11 min in 50% CPA at 4° C., and 11 min in 100% CPA at 4° C.; for medium CPA loading duration, 10 min in 20% CPA at 21° C., 10 min in 50% CPA at 4° C., and 10 min in 100% CPA at 4° C.; for long CPA loading duration, 15 min in 20% CPA at 21° C., 35 min in 50% CPA at 4° C., and 5 min in 100% CPA at 4° C. α=1.6 and β=0.005 for DMSO was used to calculate Jₜₒₓ.

Measurement of Cooling and Warming Rates

A type T thermocouple (COCO-002, OMEGA, Norwalk, Conn.) and an oscilloscope (DS1M12, USB Instrument) were used to measure the cooling and warming rates of various VR approaches. During the cryomesh VR, the thermocouple was attached to the cryomesh, and islets were added such that the thermocouple junction was in contact with the islets. The temperature was recorded during the convective cooling and warming processes. During the cryotop VR, the thermocouple was placed in the 2 μL droplet to measure the temperature rates of change. During the copper dish cooling and convective warming, the thermocouple junction was placed at ˜1 mm from the surface of the copper dish, as previously described in Zhan, L. (2021). The 2 μL droplet was dropped onto the thermocouple, and the temperature profile at the top of the droplet was recorded. Cooling and warming rates were calculated based on the temperature profile ranging from −140° C. to −20° C. Notably, the measured sample will be in a glassy phase at −140° C.

Islet Cryopreservation

Conventional Cryopreservation (Slow Cooling)

Islets were cryopreserved via a slow freezing approach following the previously established protocol by Rajotte et al. (1984, 1989 and 1999). At 21° C., DMSO was added to the islet suspension to achieve a final concentration of 2M in a cryovial. After a 25-minute incubation with DMSO, the cryovial was placed in a −7.5° C. ethanol bath for 5 minutes. A metal rod was chilled in liquid nitrogen and used to seed ice by touching the suspension in the cryovial. After 15 minutes of latent heat of fusion release, the cryovial was cooled at 0.25° C./min to −40° C. using a control rate freezer (Kryo 560, Planer Limited, Middlesex, UK), then plunged into liquid nitrogen. Thawing was achieved using a 37° C. water bath. The thawed islets were placed in 0.75 M sucrose for 30 minutes at 0° C. to remove the intracellular CPA.

Cryomesh VR

The full-strength CPA used was 22 wt % EG+22 wt % DMSO prepared in RPMI medium. To load the CPA, islets were first incubated in 20% CPA (i.e., 4.4 wt % EG+4.4 wt % DMSO) for 10 min at 21° C., followed by 50% CPA for 10 min at 4° C. and in 100% CPA for 10 min at 4° C. The islets suspension was then transferred to the cryomesh placed on a wicking material (i.e., paper towel). The CPA cocktail was wicked away through the nylon mesh and islets remained on the cryomesh. Clumping of islets was avoided. The cryomesh was quickly plunged into liquid nitrogen and stored in a liquid nitrogen tank. To thaw the islets, the cryomesh was plunged rapidly into the rewarming solution consisting of 11 wt % EG, 11 wt % DMSO, and 5 wt % sucrose at 4° C. for CPA removal. After 10 min, the rewarming solution was diluted 2-fold using ice-cold 10 wt % sucrose solution, and the islets were incubated for another 10 min at 4° C. The islets were then transferred to 21° C., and the suspension was diluted 2-fold using 10 wt % sucrose solution. After 5 min, the islets were placed back in RPMI medium for 15 min as the last CPA removal step. At this time, all islets detached from the nylon mesh.

Cryotop VR

Using the same CPA loading procedure as in the cryomesh VR approach, 10-20 islets were included in a 2 μL droplet and placed on the cryotop. The cryotop was quickly plunged into liquid nitrogen for vitrification. The rewarming and CPA removal process was the same as that performed in the cryomesh VR.

Copper Dish Cooling & Convective Warming

A 2 μL droplet, including 10-20 islets, was dropped onto a copper dish floating in liquid nitrogen for vitrification. The vitrified droplet was convectively rewarmed in the unloading solution. The CPA removal steps were kept the same as in the cryomesh VR approach.

Copper Dish Cooling & Laser Nanowarming

Gold nanorods (nanoComposix, San Diego, Calif.) were added to the droplet to reach the concentration of 2.8×10¹⁰ parts/ml. After vitrifying the islet and gold nanorod embedded droplet (2 μL) on the prechilled copper dish, a 1064 nm pulse laser was used to rewarm the droplet. The pulse length is 7.5 ms, laser voltage is 250 V. A high-speed camera (Q1v, nac Image Technology, Salem, Mass.) was used to record the laser warming process at 4000 frames per second.

Transmission Electron Microscopy (TEM)

Islets were analyzed by TEM as previously reported in Zhan, L., Nat. Commun. (2021) with slight modifications. Briefly, islets were fixed in 2.5% glutaraldehyde in 0.1 M sodium cacodylate buffer (pH 7.4) at 4° C. overnight and post-fixed in 2% aqueous osmium tetroxide for 1 hour, then dehydrated in gradual ethanol (30% to 100%) and propylene oxide, embedded in Epon812 and cured for 48 hours at 60° C. 65 nm uhrathin sections were collected onto 200 mesh copper grids, and stained with uranyl acetate (15 min) and lead citrate (2 min) before examination by transmission electron microscopy. Images were captured with a FBI Tecnai G2 Spirit Transmission electron microscope (ThermoFisher).

Viability Assessment

Qualitative measurement of intact islet viability was performed using Acridine Orange (AO) and Propidium Iodide (PI). Intact islets were stained with 8 ng/mL AO and 20 ng/mL PI (Sigma) for 2 minutes at room temperature, coverslipped, and imaged using an Olympus Fluoview 3000 inverted confocal microscope (Olympus, Center Valley, Pa.) with 502/525 nm filters for AO and 493/636 nm for PI. The images were captured at 4,020×4,020-pixel resolution using a 20× magnification objective.

Quantitative viability was measured on dissociated islet cells. After islet treatment, islets were incubated in a dynamic culture flask at 70 RPM, 37° C., and 5% CO₂ for three hours in S3 media. The islets were dissociated into single-cell suspensions in TrypLE Express (Thermo Fisher Scientific; Cat #12605010), quenched with S3 containing FBS, and stained with 8 ng/mL AO plus 20 ng/mL PI. After 15 seconds of incubation, 10 μL of the suspension was pipetted onto the Countess Cell Counting Chamber Slides (Thermo Fischer Scientific; Cat #C10228), and viability was quantified using a Countess II FL cell counter (Invitrogen by Thermo Fisher Scientific; Cat #AMQAF1000). The accuracy of the dissociated quantitative viability measurement technique was validated by comparing values to those obtained by image analysis of 3D reconstructions of confocal images of AO/PI stained intact islets.

Mitochondrial Membrane Potential Measurements

Before performing the assay, islet clusters received were incubated in a dynamic culture flask at 70 RPM, 37° C., and 5% CO₂. The islets were stained with 25 μL of 50 ng/mL reconstituted Tetramethylrhodamine ethyl ester, perchlorate (TMRE) (Biotium #70005) at room temperature for 1 minute and 45 seconds on microscope slides. Following this, glass coverslips (Chase Scientific ZA0294) were placed over the islets and imaged using an Olympus Fluoview 3000 confocal inverted microscope (excitation/emission filters: 594/574 nm). The images were captured at 4,020×4,020 resolution using a 20× magnification objective. The membrane potential of the islet was quantified by measuring fluorescence intensity using Olympus CellSens Dimension software (v1.17).

ATP Measurement

Islets were incubated in a dynamic culture flask at 70 RPM, 37° C., and 5% CO₂ before each assay. Standard-sized islets (˜150 μm) were handpicked, and 3 IEQ/well were placed in each well of black 96-well plates (Greiner Bio-One; Cat #89131-680) in 50 μL of RPMI at 37° C. 50 μL of prewarmed Promega CellTiter-Glo 3D cell viability reagent was added to each well. ATP standard (Roche) was used as positive control and to calibrate the results. The plate was sealed, covered with aluminum foil, and placed on an orbital shaker at room temperature for 5 minutes at 80 RPM. The plate was then incubated at room temperature for 25 minutes without shaking. A BioTek Synergy 2 Multi-Mode Microplate Reader (BioTek, Winooski, Vt.) was used to capture the luminescence, analyzed using the BioTek GenS software, and reported as relative light units (RLU)/IEQ.

Cellular Respiration/Oxygen Consumption Rate (OCR) Measurement

Islets were incubated in a dynamic culture flask at 70 RPM, 37° C., and 5% CO₂ for 3 hours before the assay was performed. Cellular respiration was measured using the Agilent Seahorse XF Mito Stress Test and Agilent SeaHorse XFe24 Islet Capture FluxPak (Agilent #103418-100) plates and grids. Islets were handpicked into wells containing 500 μL of culture media in sufficient numbers to cover 50% of the inner circle of each sample well. The islet capture screen was carefully and securely fit onto the plate. Islets were washed twice with SeaHorse media (SeaHorse XF DMEM (Agilent #103575-100) supplemented with 1 mM pyruvate, 2 mM glutamine, and 5.6 mM glucose, and equilibrated for 1 hour at 37° C. Assay reagents were loaded in a previously hydrated sensor cartridge. The assay plate was inserted into a calibrated Agilent SeaHorse XFe24 analyzer, and the Mito Stress test was performed according to the manufacture's protocol with the following optimized reagent concentrations: mouse islets [5 μM Oligomycin, 1 μM Carbonyl cyanide 4-trifluorometheoxyphenylhydrazone (FCCP), 10 μM each Rotenone and Antimycin A], SC-beta islets [10 μM Oligomycin A, 2 μM FCCP, 10 μM each Rotenone and Antimycin A], human islets [50 μM Oligomycin A, 10 μM FCCP, 10 μM each Rotenone and Antimycin A], and porcine islets [50 μM Oligomycin A, 2.5 μM FCCP, and 10 μM each Rotenone and Antimycin A]. OCR values were obtained over 200 minutes. If the sampling rate differed between plates, the time course was scaled over the standard observation period.

For normalization between wells, the islets in each well were lysed in 1M Ammonium Hydroxide+0.2% Triton X-100. The DNA content was measured by PicoGreen assay (Molecular Probes, Eugene, Oreg.) using standardized calibration controls for quantification on a BioTek Synergy 2 Multi-Mode Microplate Reader (BioTek). Individual cellular respiration parameters, including OCR for basal respiration, ATP production, proton leak, maximal respiration, spare respiratory capacity, and nonmitochondrial respiration, were calculated as described in Yépez, V. A., et al. (2018) and normalized to the DNA content obtained for individual wells.

TUNEL Stain

Islet clusters received were incubated in a dynamic culture flask at 70 RPM, 37° C., and 5% CO₂ before the assay was performed. According to the manufacturer's protocol, apoptotic cells were stained using ApopTag Peroxidase in situ Apoptosis Detection Kit (Milipore Sigma; Cat #S7100). Following staining and washing, the slide was mounted by dehydrating with xylene, and a coverslip applied and mounted using mounting media. The number of apoptotic cells in each islet was counted under a light microscope.

Annexin Stain

Islet clusters received were incubated in a dynamic culture flask at 70 RPM, 37° C., and 5% CO₂ before the assay was performed. 5×Annexin V Binding Buffer (Biotium; Cat #99902) was diluted in dH₂O to obtain 1×Binding Buffer. Islets were washed twice using 1× Binding Buffer. The staining solution was prepared by diluting Annexin V conjugate in 1× Binding buffer to a final concentration of 2.5 μg/ml and incubated with the islets at room temperature for 15-30 minutes protected from light. The islets were then washed with 1×Binding Buffer 3 times and imaged within 30 minutes using the Olympus Fluoview 3000 confocal inverted microscope with an excitation/emission of 490/515 under a 20×objective. The Ca²⁺ phospholipid-binding protein with a high affinity for phosphatidylserine was quantified by gating the false-color mapping fluorescence using Olympus CellSens Dimension software (v1.17) and statistically analyzed.

Glucose Stimulated Insulin Secretion (GSIS) Assay

GSIS assays were conducted as described in Veres, A., et al. (2019). Briefly, islets were washed twice in low-glucose (3.3 mM glucose) Krebs Ringer (KRB) buffer (128 mM NaCl, 5 mM KCl, 2.7 mM CaCl₂, 1.2 mM MgSO₄, 1 mM Na₂HPO₄, 1.2 mM KH₂PO₄, 5 mM NaHCO₃, 10 mM HEPES, 0.1% FAF-BSA in DI water). The islets were then loaded into 24-well transwell inserts (Millicell cell culture insert, PIXP01250) and fasted in low glucose KRB for 1 hour at 37° C. Islets were washed once in low-glucose KRB and then incubated in low-glucose KRB for 1 hour at 37° C. After incubation, the supernatant was collected and stored at −20° C. until analysis. The islets were then transferred to high-glucose KRB (16.7 mM) for 1 hour at 37° C., and the supernatant was collected and stored. They were then transferred to low-glucose KRB with 30 mM KCl to observe depolarization conditions. The islets were incubated in this buffer for 1 hour, and the supernatant was collected. Finally, the islets were dispersed via incubation with TrypLE and counted using a Countess automated cell counter (ThermoFisher). Collected supernatants were analyzed by ELISA for human insulin concentrations (ALPCO, 80-INSHUU-E01.1) and normalized for cell number. Mouse islet insulin was measured by HTRF assay (Cisbio PerkinElmer, Codolet, FR; Cat #62IN1PEG).

Syngeneic Mouse Islet Transplantation and Intraperitoneal Glucose Tolerance Test (IPGTT)

C57BL/6 mice (6-8 weeks old males, Charles River Laboratories) were rendered diabetic by single-dose (220 mg/kg) streptozotocin (Sigma; Cat #S0130) intraperitoneal injection. Blood glucose levels were measured after 4-7 days, and diabetes was confirmed by two successive daily measurements of >400 mg/dL. Marginal mass islet transplants of 250 islets per recipient were performed under the recipient mouse's left kidney capsule as previously described in Tang, Q., et al. (2004). Blood glucose (BG) measurements were made daily. Transplant success was measured on the first day of two successive daily measurements of BG<200 mg/dL. Graft failure was defined as the first day of two consecutive measurements >250 mg/dL.

On posttransplant day 50, IPGTT was performed by fasting mice for 16 hours (overnight) and then injecting 2.5 g/kg glucose IP and measuring BG every 15 minutes for 150 minutes.

To confirm that the transplanted islets were controlling BG levels and not a restoration of native beta cell function, left nephrectomy (including islet graft) was performed for a subset of transplants at posttransplant day 60, and return of hyperglycemia was verified. Day 60 explants were fixed and stained for insulin and glucagon.

Xenogeneic Islet Transplantation and IPGTT

Human SC-beta and porcine islets were transplanted under the kidney capsule of non-diabetic male NOD-scid-I12rgc^{-/-} (NSG) (Jackson Laboratory, Bar Harbor, Me.) mice (age >8 weeks) as previously described in Pagliuca, F. W., et al. (2014). Briefly, islet clusters containing a total of 5×10⁶ cells were injected under the kidney capsule of male NSG mice. Control mice underwent a mock surgery where saline was injected into the kidney capsule. Post-surgery, mice were singly housed and monitored until the end of the study. Posttransplant facial bleeds were performed at weeks 4, 8, and 12 to measure human insulin levels. At 14 weeks, posttransplant modified IPGTT was performed. Mice were fasted for 16 hours, and blood was collected before and 30 min after intraperitoneal injection of 2.5 g/kg glucose bolus. Serum was separated from blood using microvettes (Sarstedt; 20.1292.100), and human insulin was quantified using the Human Ultrasensitive Insulin ELISA (ALPCO, 80-INSHUU-E01.1). Following IPGTT, the graft containing kidneys were explanted and stained for insulin and glucagon.

For porcine islet xenotransplants, 350 islets were transplanted under the kidney capsule of non-diabetic NSG mice (male or female, age >8 weeks). At 60 days posttransplant, the graft-containing kidneys were removed, fixed, and stained for insulin and glucagon.

Insulin and Glucagon Immunofluorescence Labeling

Recovered kidneys with transplanted islets under the kidney capsule were fixed in 70% Alcoholic Formalin (BBC Biochemical; Cat #0460) and embedded in paraffin blocks. 5 μm sections were cut using a microtome. The sections were deparaffinized using xylene and decreasing 100%, 90%, 80%, and 70% EtOH concentrations. Slides were incubated with blocking buffer (5% Bovine Serum Albumin (Millipore Sigma; Cat #B6917)) in DPBS with Ca²⁺ and Mg²⁺ (Thermo Fisher Scientific; Cat #14040141) for 20 minutes and then stained with species-specific reagents as follows.

Syngeneic Mouse Islet Transplants

Kidney sections were incubated at room temperature with FLEX Polyclonal Guinea Pig Anti-Insulin Antibody (Agilent; Cat #IR002) for one hour at room temperature. The sections were gently washed 5 times using the blocking buffer and then incubated with Recombinant Rabbit Anti-Glucagon antibody (Abcam; Cat #ab92517) in blocking buffer (1:570) for 1 hour. The sections were then washed 5 times gently with blocking buffer and incubated with Goat anti-Guinea Pig IgG (H+L) Secondary Antibody (Alexa® Flour 488) (Abcam; Cat #ab150185; 1:250) and Goat Anti-Rabbit IgG H&L (Alexa® Flour 647) (Abcam; Cat #ab150079) in blocking buffer (1:250) for 1 hour at room temperature. After gently washing the sections with 4° C. DPBS with Ca²⁺ and Mg²⁺, the sections were labeled with 4′,6-diamidino-2-phenylindole DAPI (Sigma-Aldrich; Cat #F6057), coverslipped (Chase Scientific ZA0294), and placed at 4° C. for 2 hours before imaging.

Porcine Islet and SC-Beta Xenotransplants

Kidney sections were incubated at room temperature with Mouse Monoclonal anti-insulin antibody [K36aC10] (Abcam; Cat #ab6995) in blocking buffer (1:50 dilution) and Recombinant Rabbit Anti-Glucagon antibody (Abcam; Cat #ab92517) in blocking buffer (1:570) for 1 hour at room temperature. The sections were then rinsed 5 times gently with blocking buffer and incubated with Goat Anti-Mouse IgG H&L (Alexa Flour® 647) (Abcam; Cat #ab150115) in blocking buffer (1:250) and Goat Anti-Rabbit IgG H&L (Alexa® Flour 488) (Abcam; Cat #ab150077) in blocking buffer (1:300) for one hour at room temperature. After gently washing the sections with 4° C. DPBS with Ca²⁺ and Mg²⁺, the sections were labeled with 4′,6-diamidino-2-phenylindole DAPI (Sigma-Aldrich; Cat #F6057), coverslipped (Chase Scientific ZA0294), and placed at 4° C. for 2 hours before imaging.

An Olympus Fluoview 3000 confocal inverted microscope was used for imaging.

Statistics

Statistical analysis was performed in R version 4.0.3 (R Foundation for Statistical Computing, Vienna, Austria). Normality was tested with the Shapiro-Wilk test for continuous variables or graphically using qq plots and distribution histograms. Homogeneity of variance was assessed using Levene's test. For normal or near-normal group comparisons, ANOVA testing with pairwise post hoc Tukey HSD test was used to determine statistical differences. For groups with unequal variance, the Games-Howell test was used. Non-normal variables were tested using the non-parametric Kruskal-Wallis test for overall significance and the pairwise Wilcox (Mann Whitney U) test for individual group comparison. P values were adjusted for multiple comparisons using the Benjamini-Hochberg method. Time to event analysis was performed using the Kaplan Meier method with the log-rank test of significance. Data are presented as mean±standard deviation (s.d.) unless specifically detailed otherwise. A P value of <0.05 was considered significant. R version 4.0.3 was used for statistical analysis. MATLAB 2018b was used for calculating volume changes of islets from the experimentally recorded shrink-swell videos. MATLAB 2019a was used for the shrink-swell curve fitting and chemical toxicity cost function evaluation.

Results

Preparation of Islets

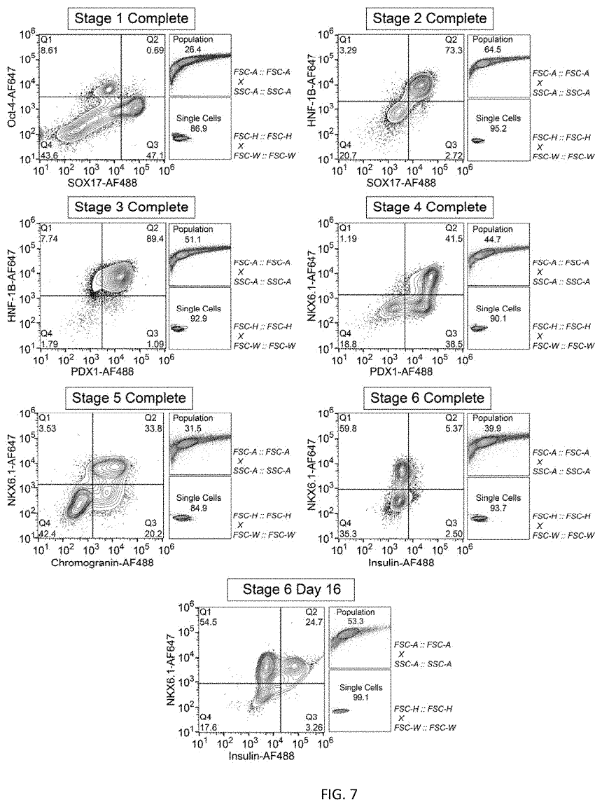

Cryopreservation and post-warming function of mouse, porcine, human, and human SC-derived (SC-beta) islets was tested. The SC-beta islets used were beta cell clusters generated by differentiation of a human embryonic stem cell line (HUES-8) in 6 stages using non-genetic programming while in 3D suspension culture. FIG. 7 shows an example of SC-beta cell-specific characterization during each stage of differentiation. Mouse islets and SC-beta islets were used for initial development, and the approach was then validated using human and, to a more limited extent, porcine islets.

Development of a Scalable Cooling and Rewarming Method for VR

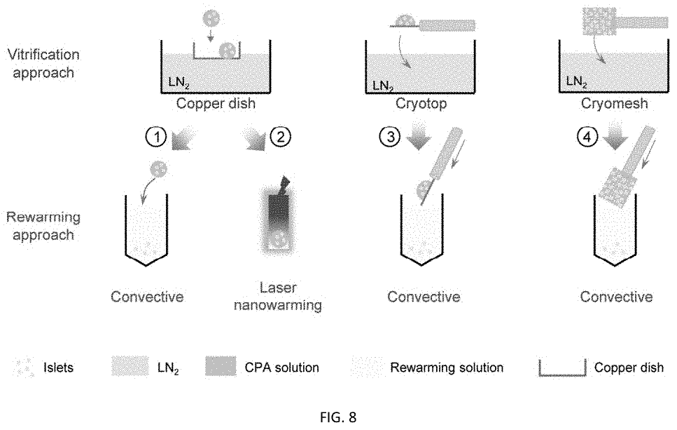

Various VR approaches were screened and assessed for their initial performance in cooling, rewarming, and scalability. Three commonly used droplet-based VR strategies were compared: 1) copper dish cooling and convective warming, 2) copper dish cooling and laser nanowarming (gold nanorods were added to the droplet to generate heat upon laser irradiation), and 3) convective cooling and warming using a cryotop device (FIG. 8; details in the Methods section). Briefly, 10-20 islets in a 2 μL droplet were vitrified and subsequently rewarmed. The cooling and warming rates were assessed by direct measurement using a thermocouple or by estimation via modeling (Khosla, K. et al. (2019)). For approaches 1-3, the cooling rates were similar: 1.1×10⁴, 1.1×10⁴, and 1.4×10⁴° C./min. The warming rates were 1.2×10⁴, 31.3×10⁴ (by modeling), and 2.3×10⁴° C./min, respectively (Table 2). In addition, 56%, 62%, and 55% post-thaw viability was obtained using approaches 1-3, respectively. Approach 1 (convective rewarming) had the slowest rewarming rate but more potential for scaling. In approach 2 (laser rewarming), the vitrified droplet was rapidly melted within the 7.5 milliseconds laser pulse. Laser nanowarming, however, is not readily scalable due to decreasing viability in larger droplets because of a droplet lensing effect, and because of procedural complexity and high cost, which would hinder clinical deployment. Approach 3 (cryotop cooling and convective rewarming) was intermediate in both rewarming and scalability. Each of these approaches suffered from suboptimal viability and potential challenges in scalability.

TABLE 2
Comparison of Vitrification-Rewarming (VR) approaches

| | Droplet | Cooling rate | Warming rate | Viability |
|---|---|---|---|---|
| VR approach | volume (μl) | (×10⁴° C./min) | (×10⁴° C./min) | (%) |
| Copper dish | 2 | 1.1 ± 0.2 | 1.2 ± 0.2 | 56 ± 14 |
| vitrification + | | | | |
| convective | | | | |
| warming ^{a} | | | | |
| Copper dish | 2 | 1.1 ± 0.2 | 31.3 | 62 ± 6.4 |
| vitrification + | | | | |
| laser nano- | | | | |
| warming ^{b} | | | | |
| Cryotop | 2 | 1.4 ± 0.2 | 2.3 ± 0.6 | 55 ± 8.6 |
| vitrification + | | | | |
| convective | | | | |
| warming ^{c} | | | | |
| Cryomesh | NA | 5.3 ± 1.5 | 30.9 ± 6.5 | 92.1 ± 1.2 |
| vitrification + | | | | |
| convective | | | | |
| warming ^{c} | | | | |

^{a} Cooling and warming rates are measured via a thermocouple. The thermocouple location is selected to represent the slowest rate inside the droplet (i.e., cooling rate measured at the top of the droplet, warming rate measured at the center of the droplet.
^{b} The warming rate is the modeling results based on Khosla et al, Langmuir, 2018, assuming islet diameter is 150 μm.
^{c} The cooling and warming rates are measured via a thermocouple.

To overcome the limitations of the droplet-based approaches, a cryomesh system was tested. The cryomesh consists of a nylon mesh (38 μm pore size) attached to a plastic handle. After loading CPA in islets while in suspension, the islets were transferred to a cryomesh support, and excess CPA cocktail was removed by wicking—an important step, as it reduces the bulk of the thermal mass in the system, leaving a thin layer of islets on the cryomesh surrounded by a minimal volume of CPA (FIG. 1B). By removing this excess fluid, the cooling and warming rates are increased by roughly an order of magnitude to 5.4×10⁴ and 30.9×10⁴° C./min, respectively, during simple convective plunging in liquid nitrogen (LN₂) for cooling and in CPA unloading solution for rewarming. CPA removal also increases the density of islets within the system vs. a droplet. For instance, a tightly packed monolayer of islets on a 2 cm×2 cm nylon mesh (i.e., 1.7×10⁴ islets) is >95% of the volume on the cryomesh, whereas 20 islets in a 2 μL droplet occupy only 1.8% of the total volume. Table 2 summarizes the performance characteristics of cryomesh, droplet, and cryotop VR approaches, emphasizing both the improved cooling and warming rates and the ability to scale up without sacrificing procedure simplicity with the cryomesh.

CPA Loading and Unloading Protocol

The CPA formulation which would avoid ice formation while minimizing toxicity was identified. Through a correlation between CPA concentration and CWR, the minimal CPA concentration in the interior of an islet was estimated and should be 42-44 wt % to avoid devitrification (ice formation) during rewarming at 30.9×10⁴° C./min using the cryomesh (FIG. 1D). CPA toxicity can occur from both chemical and osmotic injury. To avoid injury, stepwise CPA addition was tested to minimize osmotic damage (i.e., extreme volume shrinkage below 60% or volume swelling above 153%) during loading and unloading, and also assessing chemical toxicity as a function of step durations for a variety of CPA formulations.

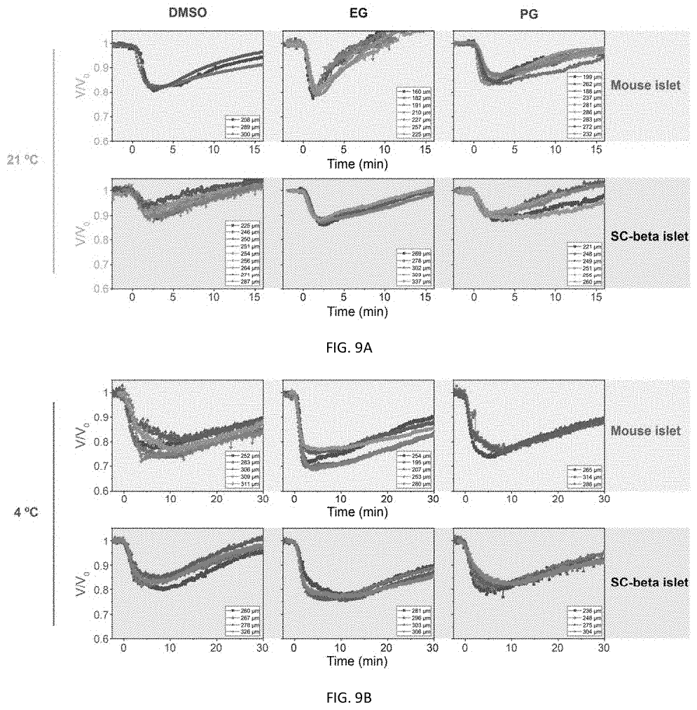

FIGS. 2A-2H show islet biophysical property measurements and CPA loading/unloading protocol design. To develop an optimized protocol for loading and unloading a given CPA, a microfluidic device was fabricated to measure islet biophysical parameters, including osmotic inactive volume (V_{b}), membrane water permeability (Lₚ), and CPA permeability (co). These measures were used to develop models that predict an optimal balance of efficiency in CPA loading while reducing both osmotic and chemical toxicity. V_{b} was measured by recording the final equilibrium volume of islets in various concentrations of hypertonic NaCl solution (FIG. 2B, lower panel). In FIG. 2B, the top panel, when subjected to 15 wt % DMSO at 4° C., the islet first shrinks and then swells. Upon exposure to hypertonic CPA, water exits the cells, and the islet shrinks. CPA then diffuses across the cell membranes, followed by water re-entering the cell, leading to swelling back towards their initial state. In the lower panel, the islet remained shrunk in NaCl solution as the cells are impermeant to salt. The scale bar is 100 μm. In FIG. 2C, Boyle-van′ t Hoff plots of mouse and SC-beta islets were used to extrapolate the osmotic inactive volume (Y_{b}) of islets (n=4-8). The Boyle-van′ t Hoff plots indicate that the osmotically inactive volume for mouse and SC-beta islets were 0.5 and 0.4, respectively (FIG. 2C). FIGS. 9A-9B show measurement of islet volume change in 15 wt % CPA cocktails. The membrane permeability parameters (Lₚ, co) were determined by recording the characteristic shrink-swell behavior of islets upon exposure to CPA cocktails containing 15 wt % propylene glycol (PG), ethylene glycol (EG), and dimethyl sulfoxide (DMSO) at different temperatures (4° C. and 21° C., FIGS. 9A-9B). FIG. 9A shows, at 21° C., the volume response of mouse islet (upper row) and SC-beta islet (lower row) to DMSO (left column), EG (middle column), and PG (right column) FIG. 9B shows, at 4° C., the volume response of mouse islet (upper row) and SC-beta islet (lower row) to DMSO (left column), EG (middle column), and PG (right column). The legends represent the diameter of islets.

The “shrink” occurs due to high water permeability upon initial CPA exposure, driving water efflux from the islets. This shrink is followed by a “swell” as permeable CPA diffuses across the cell membrane and water slowly re-enters the islets (FIG. 2B, upper panel). FIG. 2D shows normalized volume of mouse and SC-beta islets vs. time demonstrating the shrink-swell behavior when exposed to 15 wt % DMSO at 21° C. (n=5). FIG. 2E is a summary table of mouse and SC-beta islets water (Lₚ) and CPA (co) permeability at 4° C. and 21° C. As shown in FIG. 2E, the water (Lₚ) and CPA permeability (co) have higher values at a higher temperature for both mouse and SC-beta islets. The different cellular compositions between mouse and SC-beta islets likely led to the difference in permeability values between the two islet types. While the above describes the shrink-swell during loading, the inverse occurs during unloading, which is a swell followed by a shrink.

With these parameters (V_{b}, Lₚ, ω), the intracellular CPA concentration and islet volume during a multi-step loading and unloading were predicted using the two-parameter model suggested by Kleinhans (Equations 1-2). To avoid osmotic injury, the volume change of the islets was controlled to within 60% (i.e., shrink) and 153% (i.e., swell) of the initial volume. To minimize chemical toxicity, a lower temperature (4° C.) was used for CPA concentrations higher than 2 M. In addition, a chemical toxicity cost function was applied to model the effect of step duration (Equation 3 in Methods). Specifically, during the 3-step loading process, the duration of each step can be adjusted to reach the same final CPA concentration in islets. FIGS. 10A-10C show modeling of CPA chemical toxicity for short, medium, and long CPA loading step durations. The protocol with a medium step duration (10 min) was found to have the lowest theoretical chemical toxicity cost function value compared with shorter or longer step durations (FIGS. 10A-10C). In FIG. 10A, for short loading, step durations of 3 min, 11 min, and 11 min were used for the 1.3 M, 3.2 M, and 6.5 M steps, respectively. The final CPA chemical toxicity value (Jₜₒₓ, far right column) was computed to be 62.5. In FIG. 10B, for medium loading, step durations of 10 min loading time were used for each 1.3 M, 3.2 M, and 6.5 M steps. The final CPA chemical toxicity value (Jₜₒₓ, far right column) was computed to be 60.2. In FIG. 10C, for long loading, the step durations were 15 min, 35 min, and 5 min for 1.3 M, 3.2 M, and 6.5 M steps, respectively. The final CPA chemical toxicity value (Jₜₒₓ, far right column) was computed to be 73.2. Note that for all 3 loading step approaches (FIGS. 10A-10C), the unloading steps were the same. For all cases, the islet volume was maintained in the safe region (i.e., 0.6<V/V₀<1.53), and the final islet CPA concentration was 6.2 M.

FIG. 2F shows stepwise loading (step 1-3) and unloading (step 4-7) of 22 wt % EG +22 wt % DMSO for islets. FIG. 2G shows modeled islet normalized volume change during CPA loading/unloading using the measured biophysical properties. The volume of both mouse and SC-beta islets remained in the safe region. FIG. 2H shows modeled CPA concentration in the mouse and SC-beta islets. After optimization, this CPA loading protocol used the following 10-minute step intervals: 21° C. at 1.3 M, 4° C. at 3.2 M, and 4° C. at 6.5 M (FIG. 2F). Sucrose was added to the unloading solution to raise the solution osmolality and avoid osmotic shock. The volume excursions of mouse and SC-beta islets remained within the osmotic tolerances (FIG. 2G). The model predicted CPA concentration to be 6.2 M inside mouse islets and 6 M for SC-beta islets (FIG. 2H).

CPA Formulation Optimization

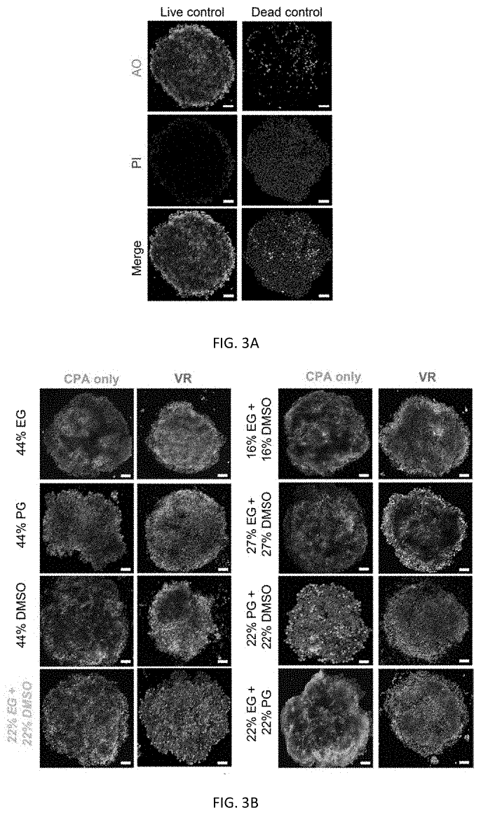

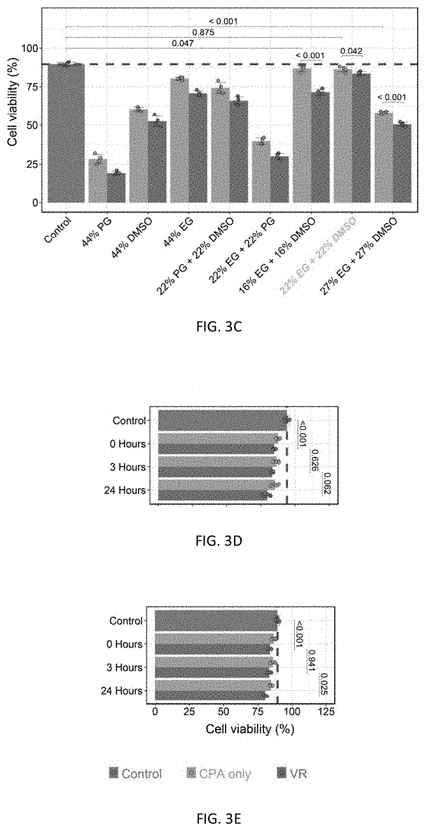

To optimize the CPA formulation, three overall CPA concentrations (33%, 44%, and 54%) were tested consisting of PG, EG, DMSO, or mixtures thereof. Qualitative and quantitative live/dead staining was used to measure SC-beta islet viability for each CPA after loading and unloading (FIG. 2F) and after VR. Qualitative measurements were determined by confocal imaging of intact islets after staining with acridine orange (AO) and propidium iodide (PI). Various CPA formulations were examined. FIGS. 3A-3E show CPA formulation optimization for high post VR viability. FIG. 3A shows confocal microscope images of live and dead controls of SC-beta islets stained by Acridine Orange (AO, cyan color) and Propidium Iodide (PI, red color). FIG. 3B shows confocal microscope images (AO/PI merge) of CPA treated (i.e., CPA loading and unloading only) and VR treated (i.e., CPA loading, VR, and CPA unloading) SC-beta islets. Fresh control islets demonstrated the expected homogenous AO⁺ staining (teal-colored cells) of live cells and only a minor fraction of PI⁺ dead cells (red) (FIG. 3A). In contrast, dead control islets (ethanol-treated) were uniformly stained red (FIG. 3A). Note that the islet diameters in all of the confocal images, here and throughout the study, are increased due to coverslip compression used to increase effective imaging depth. For quantitative measurement, CPA treated ±VR treated islets were dissociated to single cells, stained with AO/PI, and the percent of live (AO⁺/PI⁻) cells was counted (FIG. 3B). Viability measured after loading and unloading reflected the toxicity of CPA. Further decrease in viability after VR presumably reflected ice-related injury during cooling and rewarming.

FIG. 3C shows cell viability of CPA only (cyan), VR (green) treated, and live control (red) SC-beta islets. Islets were dissociated into single cells, and viability was then measured. One-way ANOVA with Tukey post-hoc test was used to compare groups and informative pairwise comparisons are shown (n=4). FIGS. 3D-3E show for mouse (FIG. 3D) and SC-beta (FIG. 3E) islet cell viability after different culture time (0, 3, 24 hrs) post-CPA only and then post-VR treatment. One-way ANOVA with Tukey post-hoc test was used to compare groups, and informative pairwise comparisons are shown (n=4). The scale bar is 100 μm. Data are individual data points and mean±s.d.

After CPA loading and unloading, EG demonstrated the least islet toxicity of the three individual components, followed by DMSO and PG (FIG. 3C). When mixtures were used at the same total concentration (44%), a mixture of EG and DMSO provided the least toxicity (cell viability was 96.4% of control), outperforming EG or DMSO alone. Islet viability also remained high (93.3% of control) after VR. This level of viability remained essentially unchanged over 24 hours of post-VR culture (FIG. 3D-3E). Decreasing the mixture concentration (i.e., to 34%) led to lower post-VR viability due to ice formation; increasing the concentration (i.e., to 54%) resulted in lower viability after CPA loading and unloading due to CPA toxicity (FIG. 3C). The best identified CPA formulation (22% EG+22% DMSO) was used throughout the study.

Viability and Morphology of Mouse, Human, Porcine, and Stem Cell-Derived Human Islets Following VR

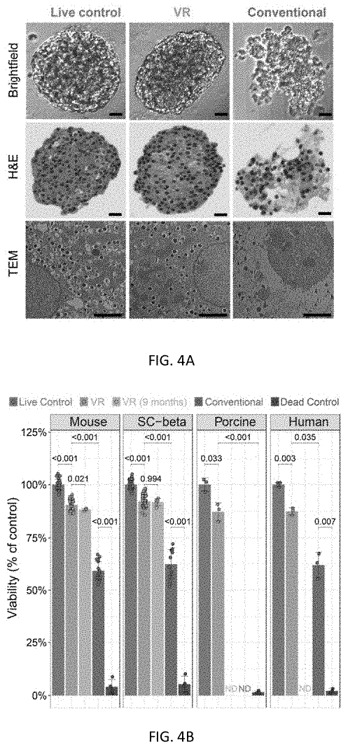

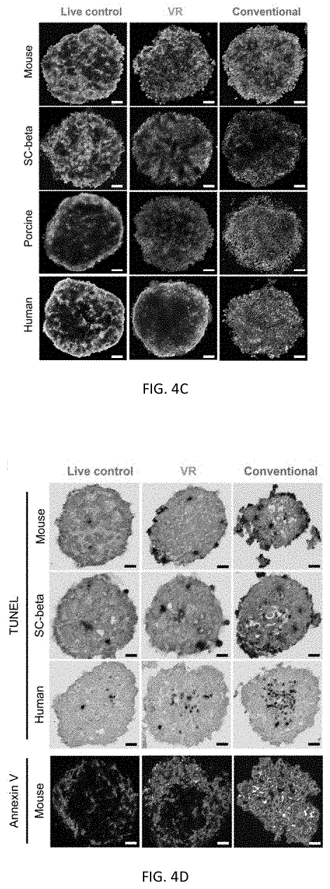

FIGS. 4A-4D show viability and morphology of mouse, porcine, human, and SC-beta islets following cryopreservation. FIG. 4A shows morphology of mouse islets from live control, VR (i.e., cryopreserved by VR), and conventional (i.e., cryopreserved by conventional slow freezing) evaluated by brightfield microscopy, H&E histology, and TEM. FIG. 4B shows viability (% of live control) of mouse, SC-beta, porcine, and human islets from treatment groups including live control, VR, VR 9 months (islets stored in LN₂ for 9 months prior to rewarming), conventional (cryopreserved by conventional slow freezing), and dead control (treated by 75% ethanol). “ND” stands for not done. One-way ANOVA with Games-Howell post hoc test was used to compare groups, and informative pairwise comparisons are shown (n=3-34/group). FIG. 4C shows confocal microscope images (AO/PI) of mouse, SC-beta, porcine, and human islets from treatment groups, including live control, VR, and conventional. FIG. 4D shows TUNEL-stained images of mouse, SC-beta, and human islets from treatment groups including live control, VR, and conventional. Bottom panel is Annexin V staining of mouse islets from the same treatment groups. The scale bars are 2 μm for TEM, 50 μm for brightfield images, 70 μm for histology and TUNEL images, and 100 μm for all fluorescence images. Data are individual data points and mean±s.d.

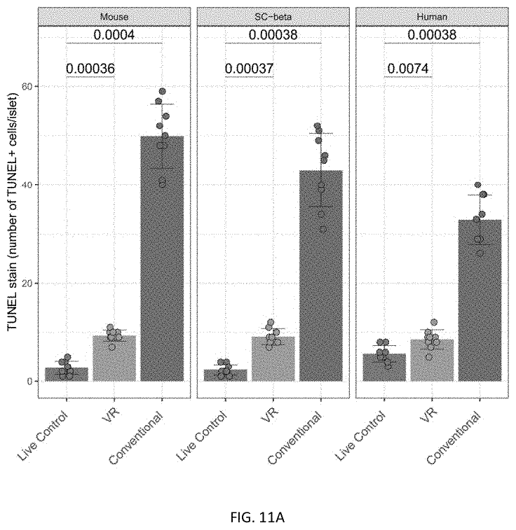

FIGS. 11A-11B show quantification of TUNEL and Annexin V translocation. In FIG. 11A, the number of TUNEL positive cells per islet was measured for mouse, human, and SC-beta islets following VR vs. conventional cryopreservation and fresh control islets (n=9/group). In FIG. 11B, Annexin V translocation to the cell surface was quantified by fluorescence intensity measurement for mouse islets of each treatment group (n=4/group). Kruskal-Wallis and pairwise Wilcox tests were used to compare groups. Data are individual data points and mean±s.d.

Using the optimized CPA formulation, loading and unloading conditions, and cooling/rewarming cryomesh system, islet morphology, viability, DNA fragmentation (TUNEL stain), and Annexin V translocation following VR were examined VR to healthy live control, dead control (ethanol-treated), and conventionally cryopreserved (i.e., slow cooling in 15% DMSO) islets (FIG. 4) were compared. Following VR, each of the 4 islet types (mouse, human, porcine, and human SC-beta) had an overall appearance and viability similar to fresh control islets and much better than conventionally cryopreserved islets (FIG. 4A). VR islets had smooth borders, rounded/oblong shape, and normal histological appearance indistinguishable from fresh islets, whereas many conventionally cryopreserved islets demonstrated disruption of normal macroscopic architecture. The differences were even more evident on ultrastructural examination using transmission electron microscopy (TEM) (FIG. 4A, bottom). TEM imaging showed that the cell and nuclear membranes, mitochondria, secretory granules, and other organelles appeared intact in VR islets. In contrast, conventionally cryopreserved islets had gross qualitative changes in cell appearance, reduced numbers of mitochondria and secretory granules, and substantial blebbing of cellular and nuclear membranes.

AO/PI staining was used to measure changes in viability associated with each islet cryopreservation technique. Qualitatively, VR islets appeared similar to live control islets, with only a slight increase in the number of red (necrotic or dead) cells (FIG. 4C) for each of the 4 islet types. Conventionally cryopreserved islets showed much more cell death (red cells). Following cryopreservation, a separate set of islets was dissociated into a single cell suspension and tested for viability on a per-cell basis (FIG. 4B). Post-VR viability, relative to control, was 90.5% for mouse, 92.1% for SC-beta, 87.2% for porcine, and 87.4% for human. The viability was unchanged over 9 months of cryopreservation (88.3% for mouse islets and 91.8% for SC-beta). Conventional cryopreservation resulted in lower viability (59.1-62.2%) than live control or VR islets.

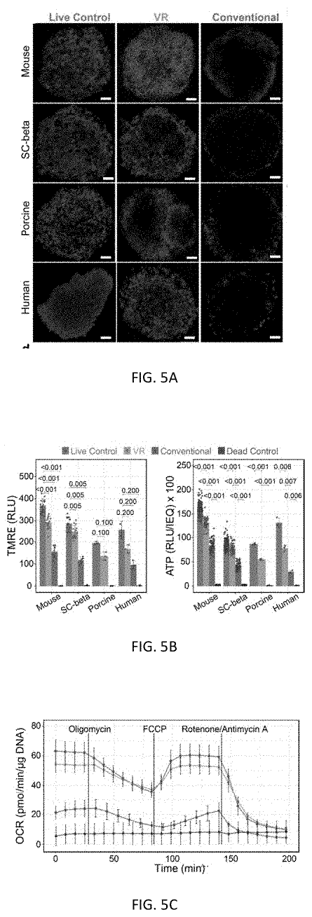

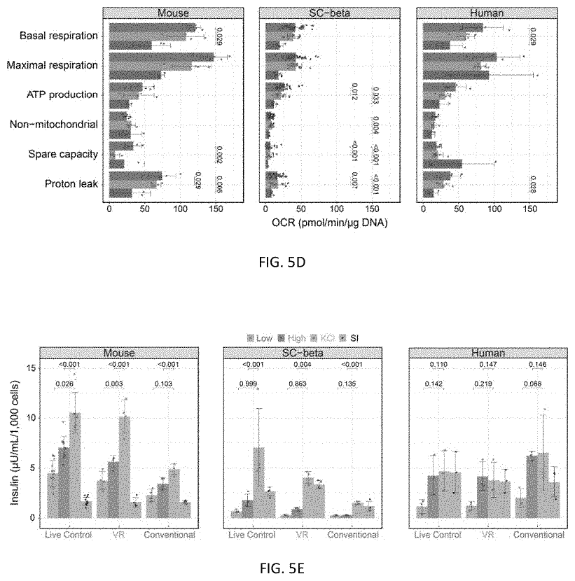

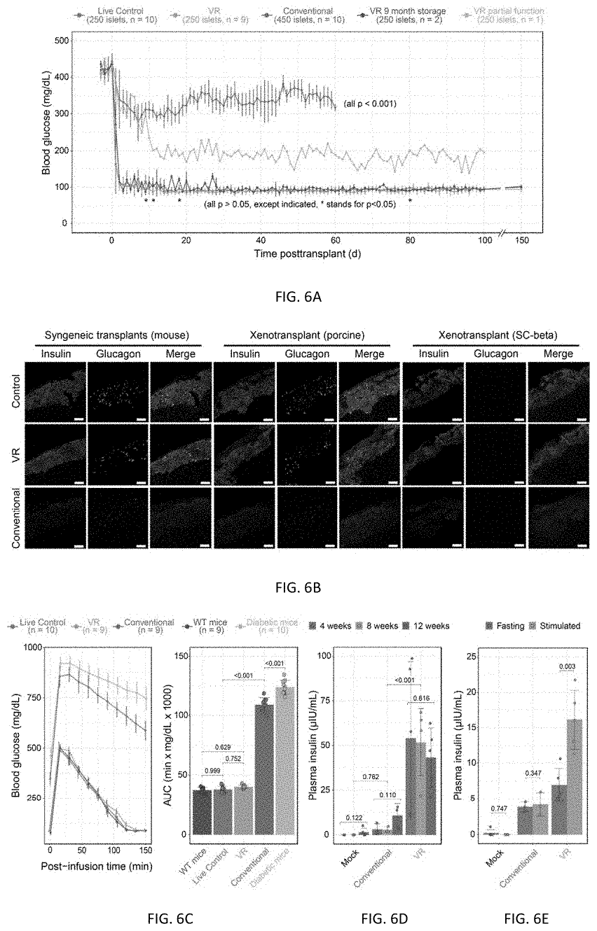

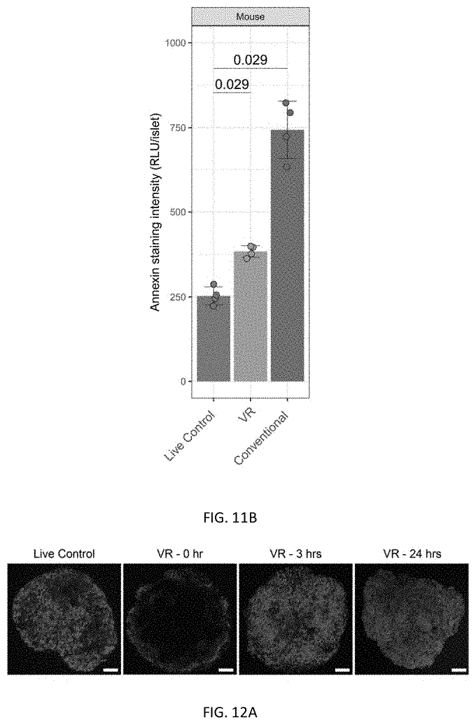

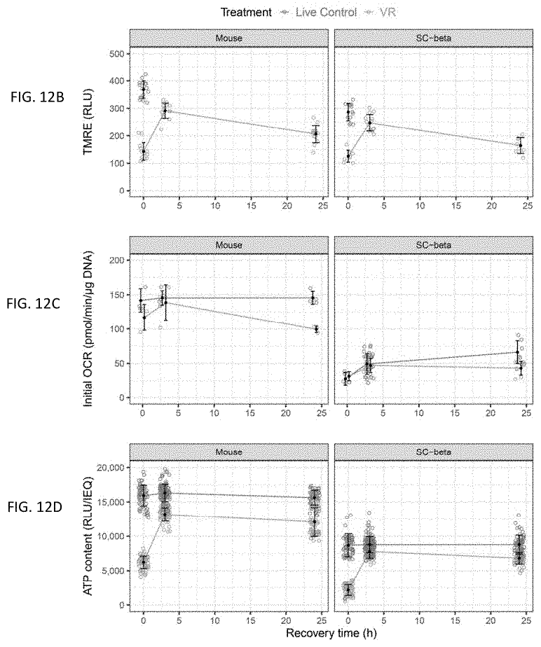

Overall islet cell viability was measured. Viability specifically in insulin expressing (i.e., beta) cells was not routinely assessed. However, similar viability between beta and non-beta cells post VR in mouse and SC-beta islets was found upon testing (FIGS. 12A-12D and FIG. 16A-16B). In addition, through the largely equivalent function of fresh control and VR islets in GSIS, transplant, and IPGTT assays, it can be assumed that the beta cell viability must be at least on par with the overall cellular viability (typically >90%). In addition, the total insulin content of VR islets as compared to fresh control was not specifically quantified, however, these groups were indirectly compared by looking at the following measures: sum of the total insulin release in GSIS assays (sum of basal insulin and that produced in response to high glucose challenge and KCl depolarization) (FIG. 5E), anti-insulin confocal images of explanted islet transplant grafts (FIG. 6B), glycemic control and IPGTT of mouse islet transplants (FIG. 6A and FIG. 6C), and in vivo glucose stimulated insulin release (FIG. 6E). Each of these measures indicated that the total effective insulin content per islet was not significantly changed by VR.

In addition to the observed low degree of cell death (PI⁺ cells) with VR islets, slightly more TUNEL⁺ and cell-surface Annexin V⁺ cells were observed after VR in comparison to fresh control islets (FIG. 4D and FIG. 11), suggesting both necrosis and apoptosis may contribute to the overall 8-12% decrease in viability with VR. Many more TUNEL⁺ and Annexin V⁺ cells were seen with conventionally cryopreserved islets. Compared with conventional cryopreservation, the VR technique described herein resulted in substantial improvements in preserving the viability and morphology of the 4 tested islet types.

Metabolic Health of VR Islets

Since viability measured by membrane permeability dyes represents a lagging indicator of islet dysfunction following treatment, other measures of islet health that could better define the expected islet function following cryopreservation were examined.

FIGS. 12A-12D show mouse and SC-beta islets metabolism recovery post VR. FIG. 12A shows confocal images of TMRE stained mouse islets. From left to right: live control, 0 hr post-VR, 3 hrs post-VR, and 24 hrs post-VR. FIG. 12B shows quantitative comparison of TMRE staining intensity from live control, 0 hr post-VR, 3 hrs post-VR, and 24 hrs post-VR islets. FIG. 12C shows initial oxygen consumption rate (OCR) of live control, 0 hr post VR, 3 hr post-VR, and 24 hr post-VR islets. FIG. 12D shows ATP content of live control, 0 hr post-VR, 3 hr post-VR, and 24 hr post VR islets. Error bars are mean±s.d. Scale bars are 100 μm.

Specifically, metabolic health, particularly islet oxygen consumption rate (OCR), is predictive of islet function in vivo. ATP levels were first examined in islets immediately post-VR and found that, at that timepoint, ATP content was significantly lower than in control islets (FIGS. 12A-12D). However, ATP levels and other metabolic measures (OCR and mitochondrial membrane potential) were largely recovered after 3 hours of culture. That timepoint was used for all further assessments.

FIGS. 5A-5E show metabolic health and in vitro function of islets following cryopreservation. FIG. 5A shows mitochondrial membrane potential (via TMRE staining) of mouse, SC-beta, porcine, and human islets from treatment groups including live control, VR, and conventional. In FIG. 5B, left panel is the quantification of TMRE staining intensity. Comparisons shown between live control and treatment groups were performed by Kruskal-Wallis and pairwise Wilcoxon tests (n=3-16/group). Right panel is the measurement of ATP levels of 4 types of islets from live and dead control groups and cryopreservation groups (i.e., VR and conventional). One-way ANOVA with Games-Howell post hoc test was used to compare groups, and informative pairwise comparisons are shown (n=3-126/group). FIG. 5C shows example oxygen consumption rate (OCR) curve showing the change in OCR during Mito Stress testing in SC-beta islets and comparing live control, VR, conventional cryopreservation, and dead control islets (n=3-4/group at each timepoint). FIG. 5D shows compilation of the metabolic OCR parameters for each islet type and each treatment group. One-way ANOVA with Tukey post hoc test was used to compare groups, and significant (P<0.05) pairwise differences are shown (n=3-33/group). FIG. 5E shows in vitro glucose-stimulated insulin secretion (GSIS) assay for mouse, SC-beta, and human islets from treatment groups including live control, VR, and conventional. One-way ANOVA with Tukey post hoc test was used to compare groups and informative pairwise comparisons are shown (n=3-12/group). The scale bar is 100 μm. Data are individual data points and mean±s.d.

Mitochondrial membrane potential in each of the 4 islet types was measured by TMRE staining and qualitative and quantitative confocal microscopy. The overall signal intensity was somewhat lower for VR islets than live control for each islet type (65.7-86.3% of control), but when adjusted for viable cell content, TMRE intensity was 75.1-93.7% of control (FIG. 5B left). The difference in TMRE intensity may be due to slower recovery in the center of the islets where there was some central lucency. This appearance appeared to improve further with 24 hours of culture (FIGS. 12A-12D). TMRE staining intensity for conventionally cryopreserved islets was 37-42% of control islets (FIG. 5B, left, statistics in the figure legend).

The number and appearance of mitochondria seen by TEM of VR islets were qualitatively similar to control islet cells (FIG. 4A, bottom). Similar to the TMRE findings, overall ATP levels in VR islets were slightly less than in the fresh control islets (FIG. 5B, right). These data suggest that cellular machinery to produce ATP remained intact, but ATP levels had not yet been fully restored to control values at the timepoint assayed.

Having shown intact mitochondria, cellular respiration was examined to assess whether VR islets were consuming oxygen to restore ATP. When compared to fresh control islets, VR islets showed a similar pattern of changes in OCR following stimulation with oligomycin (ATP synthase/complex V inhibitor), FCCP (mitochondrial membrane uncoupling agent), and a mixture of rotenone (complex I inhibitor) and antimycin A (complex III inhibitor) (FIG. 5C). Conventionally cryopreserved islets showed a dampened OCR stress response. Comparing VR to fresh control in mouse, human, and SC-beta islets, there were no differences in OCR for basal respiration, ATP production, proton leak, maximal respiration, spare respiratory capacity, and non-mitochondrial respiration (FIG. 5D). These data suggest that VR islets maintain normal metabolic function, at least as measured by cellular respiration.

In Vitro Insulin Secretion by VR Islets

In vitro glucose-stimulated insulin secretion (GSIS) testing was performed to determine if islets were functional in vitro. Mouse, SC-beta, and human islets secreted insulin in response to glucose challenge and, for mouse and SC-beta, further with complete depolarization using KCl (FIG. 5E). Porcine islets were not tested. Human islets had maximal insulin release with high glucose challenge but no further release with KCl. The stimulation indices (SI, or ratio of insulin released with exposure of high glucose concentration to that of low glucose) for VR islets of each islet type measured were not different from control islets. Conventionally cryopreserved islets also showed insulin release in response to glucose challenge, but to a lesser degree. These data confirm that VR islets are viable, metabolically active, and functional in vitro.

In Vivo Function of VR Islets

As a final measure of post-VR islet function, mouse, porcine, and human SC-beta islets were tested in syngeneic and xenogeneic mouse transplant models (FIGS. 6A-6E). FIG. 6A shows blood glucose levels of streptozotocin-induced diabetic mice after syngeneic transplant of marginal mass mouse islets (250 islets per recipient) from treatment groups including live control, VR, VR with 9-month cryopreserved storage (islets stored in LN₂ for 9 months), and conventional cryopreservation (450 islets per recipient). Statistics: All pairwise comparisons with P-value <0.05 are shown (*, P<0.05) as determined by one-way ANOVA with Games-Howell post hoc test [n=10 (control and conventional cryopreservation), 9 (VR), 1 (VR partial function), 3 (9-month storage and VR)]. FIG. 6B shows Insulin (red) and glucagon (green) staining in syngeneic (mouse) and xenogeneic (porcine and SC-beta) mouse transplant models. Treatment groups of islets include live control, VR, and conventional cryopreservation. DAPI is stained blue in the merged images. FIG. 6C shows intraperitoneal glucose tolerance testing (IPGTT) of wildtype mice, diabetic mice, and diabetic mice transplanted with live control, VR, and conventional cryopreserved islets (left panel). Area under the curve (AUC) of IPGTT (right panel). Groups are compared by one-way ANOVA and Tukey post hoc test. Only informative pairwise comparisons are shown (n=9-10/group). FIG. 6D shows xenotransplant of SC-beta islets in non-diabetic NSG mice with non-fasting plasma human insulin levels at 4, 8, 12 weeks posttransplant. FIG. 6E shows, at 14 weeks, plasma insulin level of NSG mice after fasting and 30 minutes following stimulated insulin production by intraperitoneal glucose injection. For FIGS. 6D and 6E: group comparison is by one-way ANOVA and Tukey post-hoc test with informative comparisons shown (n=3-5/treatment group). Scale bar is 200 μm. Data are individual data points and mean±s.d.

Syngeneic mouse islets (C56B1/6 to C56B1/6) transplanted under the kidney capsule demonstrated function and restored normoglycemia within 48 hours (FIG. 6A) and showed intense insulin immunofluorescence staining similar to control islets on posttransplant day 60, as did porcine and SC-beta xenotransplants in immunodeficient NSG recipients (FIG. 6B). Mouse and porcine islets also had intense glucagon staining, although potentially with a slight qualitative reduction in signal intensity compared with control islets. As expected, SC-beta transplants showed little to no glucagon staining since these had been differentiated to a beta cell lineage. Conventionally cryopreserved islets had very low insulin and glucagon staining intensity for each islet type tested.

Using a xenogeneic transplant model, the function of SC-beta islets in vivo was tested. SC-beta islets were transplanted in non-diabetic NSG mice, and random serum samples were obtained to measure human insulin at 4, 8, and 12 weeks posttransplant (FIG. 6D). VR islets secreted human insulin throughout the 12 weeks. Conventionally cryopreserved SC-beta islets had detectable human insulin production, but these levels were not statistically different from mock transplant recipients. At 14 weeks, the recipient mice were tested for fasting and stimulated human insulin production by intraperitoneal glucose injection (FIG. 6E). Conventionally cryopreserved islet transplant recipients did have detectable human insulin, but there was no increase in levels following stimulation. VR islet recipients had higher fasting levels than mock or conventionally cryopreserved islets and demonstrated a 2.3-fold increase following stimulation, confirming human SC-beta function in vivo.

Fresh control, VR, and conventionally cryopreserved mouse islets were next tested in a marginal mass (250 islets/recipient) syngeneic islet transplant model using streptozotocin (STZ)-induced diabetic recipients. Overall, VR islets rapidly restored normoglycemia in 92% (11/12) of recipients within 24-48 hours (FIG. 6A). In bivariate Kaplan Meier assessment, time to normoglycemia was not different from live control islet transplant recipients (Log-rank test, P=0.063). One VR islet recipient demonstrated only partial function, potentially due to technical issues (i.e., islet leakage from kidney capsule). Blood sugar for that recipient fell below 200 mg/dL on posttransplant day 13 and then exhibited continued moderate hyperglycemia.

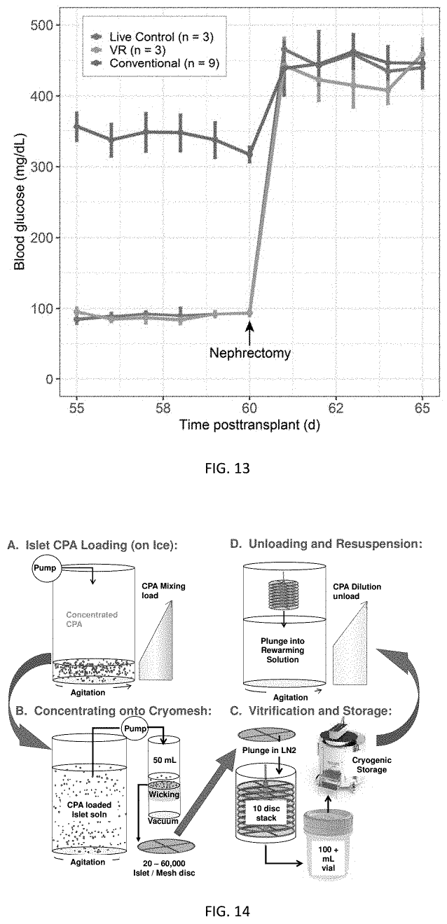

In contrast, conventionally cryopreserved islets failed to normalize blood glucose in all recipients, even with increased numbers of islets (450 islets/recipient). In FIG. 13, to validate the syngeneic transplant model, a subset of islet transplant recipients underwent nephrectomy of the islet-containing kidney on posttransplant day 60. Blood glucose rise confirmed that the transplanted islets were mainlining glycemic control and not the native pancreas. Error bars are mean±s.d. Hyperglycemia developed rapidly in all mice, confirming that the transplanted islets, not the native recipient islets, controlled blood glucose levels.

In the syngeneic transplants, glycemic control was extremely tight for VR islets. Random blood glucose levels were not higher than those of control islet transplants throughout the posttransplant course (to 150 days posttransplant). To test the result of extended cryopreservation time, we vitrified islets, stored them in LN₂ for 9 months, then rewarmed them by the VR method. These long-term stored islet transplants also demonstrated rapid blood glucose normalization and glucose stability throughout the follow-up period.

To demonstrate the level of glycemic control, intraperitoneal glucose tolerance testing (IPGTT) on posttransplant day 50 (FIG. 6C) was performed. There were no differences in the glucose response curves (FIG. 6C, left) or the areas under the curves (AUCs, FIG. 6C, right) for untreated wild-type mice, fresh control islet transplants, and VR transplants. Recipients of conventionally cryopreserved islets had a glycemic response similar to diabetic control mice.

Recovery and Scalability

Since the cryomesh system for vitrification and rewarming is intrinsically unidimensional, scaling in the x- and y-dimensions is theoretically limited only by the container geometry. VR using batches of 2,500 SC-beta islets on a 2 cm×2 cm mesh was tested. Following VR, 95% of the starting islet quantity was recovered and a viability of 89.4% (n=3) post-thaw (relative to control) was found. In the initial studies, islets at 4,250 islets/cm² were tested. To achieve clinically meaningful throughput, units of 100,000 islets could thus be preserved on 24 cm² of cryomesh.

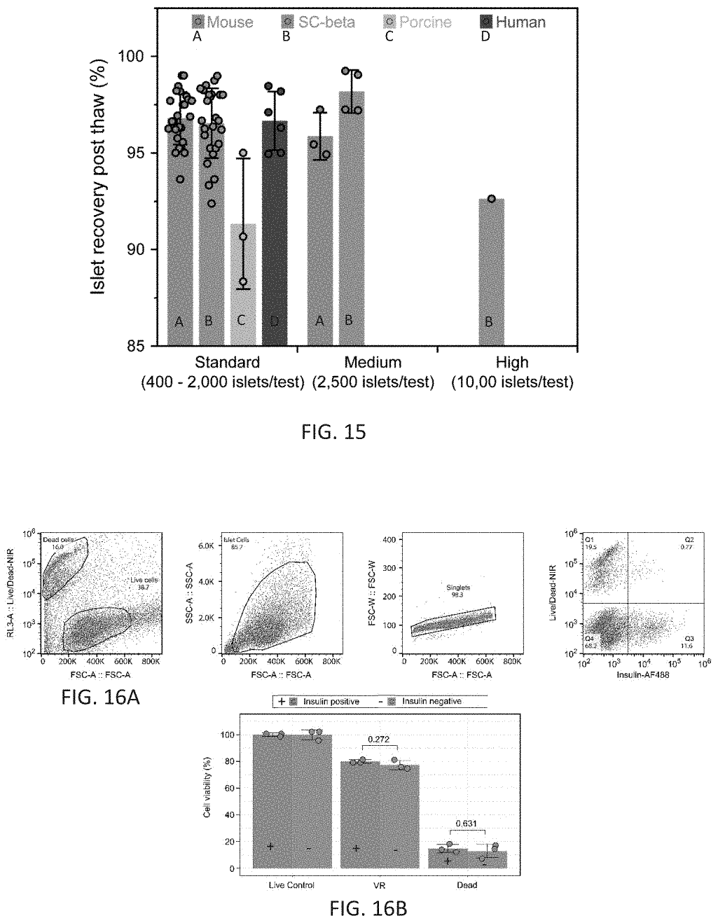

As shown in FIG. 15, islet recovery of the standard scale VR process (400-2,000 islets per test) was 96.8±1.3% for mouse (n=26), 96.6±1.7% for SC-beta (n=26), 91.3±3.4% for porcine (n=3), and 96.7±1.5% for human (n=6) islets. When it was scaled up to medium throughput (2,500 islets per test) the recovery for mouse islets was 95.9±1.2% (viability of 89.4%, n=3) and SC-beta islet recovery was 98.2±1.1% (n=4). Finally, in the initial proof-of-concept test of higher throughput SC-beta islet VR (10,000 islets per test), recovery was 92.6% (n=1).

Prophetic Example

The cryomesh VR approach is used to achieve a total throughput of 1M islet equivalents (IEQ) in increments of 60K IEQ/mesh. The viability, function, and recovery of these islets can be determined.

Engineering scale-up of the cryomesh VR approach to clinical islet quantities. An approach can be designed to increase this number in increments of 60K IEQ/mesh with semi-automated parallel processing of multiple meshes to achieve 600K IEQ/vial and multiple vials to achieve >1M IEQ. The main focus of this example is system optimization for practical engineering scale-up.

Experimental Design: A schematic outlining an example strategy for bulk cryopreservation of islets is shown in FIGS. 14A-14D. A single human donor pancreas is estimated to yield between 200,000 and 1M IEQ, with a nominal value around 350,000 IEQ. The system therefore needs to be adaptable to handle variable throughput in this range. This variability can be addressed by performing CPA loading/unloading in a bulk suspension (>250 mL solution volume) but cryopreservation on individual cryomesh discs capable of holding up to 60,000 islets each. As shown in FIG. 14A, the islets can be suspended in media and gently agitated to provide volumetric mixing. Different methods of agitation, including stirring, rocking, or orbital shaking, could be employed. A pump can be used to control the volumetric addition of concentrated CPA to provide a gradual, ramped concentration increase (e.g., 27.5% wt EG+27.5% wt DMSO added at a 4:1 ratio to achieve a final 22% wt EG+22% wt DMSO). The temperature and time of loading can be controlled to minimize CPA toxicity, as described earlier. After CPA loading, the islets can be concentrated on the cryomesh discs. Assuming 2,500 islets per cm² can be achieved (as already demonstrated), a scaled cryomesh disc (5.5 cm Diam.) can hold up to 60,000 islets. The mesh pattern, filament geometry, material, support structure, size, and shape can be engineered to optimize islet loading, rapid cooling/warming and ease of handling and storage. Islets can be concentrated on each disc in a separate container by aliquoting the loaded islet suspension and straining through the mesh and a wicking material. Once the islets are loaded and the excess CPA is drained/wicked, as shown in FIG. 14B, each disc can be vitrified and stacked in a cryogenic vial (e.g., 100-120 mL) filled with LN2 for rapid cooling and stacked mesh storage (FIG. 14C). The discs can be stacked on a central support structure to maintain spacing between the discs and allow handling as a single unit in subsequent steps. Each vial can contain up to 10 discs, which can together store up to 600,000 IEQ (and multiple vials if needed). Storage can occur in vapor or liquid nitrogen. It is estimated that roughly 648 120 mL cryogenic vials could be stored and tracked in a rack system in a standard 360L LN2 cryogenic storage freezer; however, the cryomesh disc aspect ratio and container size can be optimized for storage capacity and handling. In preparation for transplant, the matched donor islet container(s) can be retrieved from storage. Upon removal from the cryogenic vial, the entire stack of cryomesh discs can be plunged into the rewarming solution in one step, as shown in FIG. 14D. The plunging angle, orientation, speed, and gentle centrifugal movement can be engineered to ensure rapid rewarming and complete release of the islets from the mesh. A scaled-up design can be used with suitable performance characteristics for the cryopreservation of large numbers of islets.

All ranges given are intended to further include “any range there between” whether or not this is affirmatively stated.

All publications, patents and patent documents are incorporated by reference herein, as though individually incorporated by reference, each in their entirety, as though individually incorporated by reference. In the case of any inconsistencies, the present disclosure, including any definitions therein, will prevail.

Although specific embodiments have been illustrated and described herein, any arrangements that achieve the same purpose, structure, or function may be substituted for the specific embodiments shown. This application is intended to cover any adaptations or variations of the example embodiments of the invention described herein. These and other embodiments are within the scope of the following claims and their equivalents.

What is claimed is:

1. A method for cryopreservation of a biological material comprising:
identifying a cryoprotective (CPA) cocktail for vitrification of a biological material wherein the CPA cocktail comprises one or more CPAs at a vitrification CPA concentration for the biological material and wherein the identity of the one or more CPAs and/or the vitrification CPA concentration are determined by analyzing the biophysical parameters of the biological material;
loading the biological material with the one or more CPAs to attain the vitrification CPA concentration within the biological material;
transferring the CPA loaded biological material onto a cryomesh or other porous surface and removing excess CPA cocktail surrounding the CPA loaded biological material prior to cooling the CPA loaded biological material;
cooling the CPA loaded biological material on the cryomesh or the other porous surface to form a vitrified biological material;
rewarming the vitrified biological material; and
unloading the one or more CPAs from within the rewarmed biological material to eliminate the one or more CPAs from a vitrified and rewarmed (VR) biological material.

2. The method of claim 1, wherein the method minimizes osmotic stress and/or chemical toxicity to the biological material during the loading, the cooling, the rewarming and/or the unloading steps.

3. The method of claim 1 wherein the biophysical parameters of the biological material comprise inactive volume fraction, hydraulic conductivity, and/or membrane permeability to the one or more CPAs.

4. The method of claim 1, wherein the biophysical parameters determine the vitrification concentration of the one or more CPAs in the loading step and length of time to load the one or more CPAs to minimize osmotic stress and toxicity to the biological material.

5. The method of claim 1, wherein the biological material is selected from the group consisting of cell clusters, islets, human pancreatic islets, mouse pancreatic islets, porcine pancreatic islets, stem-cell derived islets, and stem-cell derived beta islets.

6. The method of claim 1 wherein the one or more CPAs in the CPA cocktail comprises ethylene glycol (EG) and dimethyl sulfoxide (DMSO).

7. The method of claim 1 wherein the loading comprises multi-steps and the concentration of the one more CPAs is increased in each successive loading step and the unloading comprises multi-steps and the concentration of the one or more CPAs is decreased in each successive unloading step.

8. The method of claim 6 wherein the unloading further comprises gradually increasing the amount of a non-penetrating CPA in the CPA cocktail.

9. The method of claim 1 wherein the cooling rate for cooling the biological material is greater than about 50,000° C./min.

10. The method of claim 1 wherein the rewarming rate for rewarming the vitrified biological composition is greater than about 200,000° C./min.

11. The method of claim 1 wherein the VR biological material has a viability of at least about 80% relative to a control.

12. The method of claim 1 further comprising transplanting the VR biological material.

13. The method of claim 1 wherein the VR biological material comprises greater than about 2500 islets with greater than about 95% recovery and greater than about 85% viability.

14. The method of claim 1 wherein the VR biological material is VR Islets and wherein the method is scalable to produce at least 100,000 islets per batch.

15. A method of scaling up production of cryopreservation of biological material comprising:
identifying a cryoprotective (CPA) cocktail for vitrification of a biological material wherein the CPA cocktail comprises one or more CPAs at a vitrification CPA concentration for the biological material and wherein the identity of the one or more CPAs and/or the vitrification CPA concentration are determined by analyzing the biophysical parameters of the biological material;
loading the biological material with the one or more CPAs to attain the vitrification CPA concentration;
transferring the CPA loaded biological material onto a cryomesh or other porous surface and removing excess CPA cocktail surrounding the CPA loaded biological material prior to cooling the CPA loaded biological material, wherein the length and the width of the cryomesh or other porous surface is extended to accommodate the CPA loaded biological material;
cooling the CPA loaded biological material on the cryomesh or the other porous surface to form a vitrified biological material;
rewarming the vitrified biological material; and
unloading the one or more CPAs from within the rewarmed biological material to eliminate the one or more CPAs from a vitrified and rewarmed (VR) biological material
wherein the biological material are cell clusters and the method produces at least about 10,000 cell clusters per batch.

16. The method of claim 15 wherein two or more layers of the cryomesh or other porous surfaces are stacked to increase the number of cell clusters per batch.

17. The method of claim 15 wherein the method produces at least about 100,000 cell clusters per batch.

18. A method of therapeutic transplantation of a biological material comprising transplanting the biological material into a patient, wherein the biological material comprises VR islets derived from one or more donors, the VR islets having a viability of at least 80 percent.

19. The method of claim 18 wherein the biological material comprises greater than about 2500 VR islets with greater than about 95% recovery and greater than about 85% viability.

20. A vitrified and rewarmed biological composition produced by the method of claim 1.
